(19) United States
(12) Patent Application Publication
(10) Pub. No.: US 2019/0313942 A1
(43) Pub. Date: Oct. 17, 2019

(54) INGESTIBLE SYSTEM TO MONITOR GASTROINTESTINAL HEALTH IN SITU

(71) Applicant: Massachusetts Institute of Technology, Cambridge, MA (US)

(72) Inventors: TIMOTHY KUAN-TA LU, Cambridge, MA (US); Mark K. Mimee, Cambridge, MA (US); Phillip Nadeau, Cambridge, MA (US); Anantha P. Chandrakasan, Belmont, MA (US)

(73) Assignee: Massachusetts Institute of Technology, Cambridge, MA (US)

(21) Appl. No.: 15/955,080

(22) Filed: Apr. 17, 2018

(51) Int. Cl. A61B 5/07; A61B 5/00; A61B 5/145

(52) U.S. Cl. CPC A61B 5/073; A61B 5/4255; A61B 5/4238; A61B 7/008; A61B 5/14539; A61B 5/6861; A61B 5/4233; A61B 5/14546

(57) ABSTRACT

Disclosed herein are novel devices comprising small, ultra-low power microelectronic components. In some instances, the microelectronic components is combined with a biosensor component that enables in situ detection of biomolecules. Also disclosed herein are methods of detecting signal analytes and methods of monitoring the health of a patient using these novel devices.

### GOVERNMENT SUPPORT

This invention was made with Government support under Grant No. CCF-1124247 awarded by the National Science Foundation, and Grant No. N00014-13-1-0424 awarded by the Office of Naval Research. The Government has certain rights in the invention.

### FIELD

Disclosed herein are novel devices comprising small, ultra-low power microelectronic components. In some instances, the microelectronic components is combined with a biosensor component that enables in situ detection of biomolecules. Also disclosed herein are methods of detecting signal analytes and methods of monitoring the health of a patient using these novel devices.

### BACKGROUND

While electronics provide a versatile interface for collecting, processing, and sharing information, their ability to directly sense biomolecules in vivo has been limited due to their dependence on labile biochemical transducers that necessitate large, power-demanding circuits for sensitive detection.

### SUMMARY

In some aspects, the disclosure relates to devices comprising small, ultra-low power microelectronic components that overcome these limitations. In some embodiments, a device comprises an electrical component wherein the electrical component comprises: at least one detector configured to charge a respective capacitance, wherein each of the at least one detector is configured to detect an output from biosensor component; a comparator configured to compare respective voltage signals from each of the at least one detector to a reference voltage, each voltage signal indicating the charge stored by the respective capacitance; an oscillation counter configured to, when the voltage signal from a first detector of the at least one detector exceeds the reference voltage, store a number of oscillator cycles taken for the first detector to charge the capacitance; and a transmitter configured to, when the voltage signals from each of the at least one detector exceed the reference voltage, wirelessly transmit the respective stored numbers of oscillator cycles taken for the at least one detector to charge the capacitance. In some embodiments, at least one of the at least one detectors is a photodetector. In some embodiments, the device contains a calibration scheme for detecting and removing background light and temperature-induced drift.

In some embodiments, the device is shaped as a capsule or spherocylinder. In some embodiments, the capsule or spherocylinder comprises a cross-sectional diameter that is shorter than 5 cm, 4.5 cm, 4 cm, 3.9 cm, 3.8 cm, 3.7 cm, 3.6 cm, 3.5 cm, 3.4 cm, 3.3 cm, 3.2 cm, 3.1 cm, 3.0 cm, 2.9 cm, 2.8 cm, 2.7 cm, 2.6 cm, 2.5 cm, 2.4 cm, 2.3 cm, 2.2 cm, 2.1 cm, 2.0 cm, 1.9 cm, 1.8 cm, 1.7 cm, 1.6 cm, 1.5 cm, 1.4 cm, 1.3 cm, 1.2 cm, 1.1 cm, 1.0 cm, 0.9 cm, 0.8 cm, 0.7 cm, 0.6 cm, or 0.5 cm. In some embodiments, the device can be swallowed by a patient.

In some embodiments, the device further comprises at least one biosensor component, wherein each of the at least one the biosensor component: is sensitive to the presence of at least one signal analyte; and communicates the presence of the at least one signal analyte to the electrical component, optionally wherein the communication is proportional to the abundance of the at least one signal analyte.

In some embodiments, the biosensor component is separated from the outside environment by a semi-permeable membrane that permits diffusion of the at least one signal analyte. In some embodiments, the semi-permeable membrane is a polyethersulfone membrane filter.

In some embodiments, at least one of the at least one biosensor component is an enzymatic biosensor or a non-enzymatic biosensor. In some embodiments, the non-enzymatic biosensor comprises an antibody, a binding protein, or a nucleic acid. In some embodiments, the enzymatic biosensor or non-enzymatic biosensor is a cellular biosensor comprising at least one microorganism. In some embodiments, the at least one microorganism is present in the device in a dormant state. In some embodiments, the at least one microorganism is combined with additional substances to aid in removing the at least one microorganism from its dormant state, to provide nutrients to the at least one microorganism, and/or to prolong the lifetime of the at least one microorganism. In some embodiments, at least one of the at least one microorganism comprises an engineered genetic circuit. In some embodiments, the output of the engineered genetic circuit is luminescence, fluorescence, ion flow, or turbidity.

In some embodiments, at least one of the at least one signal analyte is selected from the group consisting of a microorganism, a biomolecule, or an inorganic molecule. In some embodiments, at least one of the at least one signal analyte is a biomolecule. In some embodiments, the biomolecule is selected from the group consisting of heme, thiosulfate, and acyl-homoserine lactone.

In other aspects, the disclosure relates to methods of detecting at least one signal analyte. In some embodiments, a method comprises contacting a device as described above with a sample and comparing the output of the device to a control. In some embodiments, the sample is selected from the group consisting of soil, water, air, or food.

In other aspects, the disclosure relates to methods of monitoring the health of a patient. In some embodiments, a method comprises contacting a device as described above with a patient and comparing the output of the device to a control. In some embodiments, the control is established through analysis of a population of healthy patients.

In some embodiments, the contacting of the device with the patient occurs by oral administration or deposition of the device in the esophagus, stomach, or intestine. In some embodiments, the contacting of the device with the patient occurs by surgical implantation.

In some embodiments, the patient is a human patient. In some embodiments, the human patient is predisposed to a disease, disorder, morbidity, sickness, or illness. In some embodiments, the human patient has been diagnosed with a disease, disorder, morbidity, sickness, or illness.

In other aspects, the disclosure relates to ingestible devices—contained within a capsule or spherocylinder—comprising an electrical component and at least one biosensor component wherein: the electrical component comprises wireless low-power electronics powered by (a) a battery, (b) energy harvesting, or (c) wireless power transfer, wherein the low-power electronics comprise at least one detector; and each biosensor component (a) is separated from the external environment via a semi-permeable membrane, (b) is sensitive to the presence of at least one signal analyte, and (c) communicates the presence of the at least one signal analyte to the electrical component, optionally wherein the communication is proportional to the abundance of the at least one signal analyte. In some embodiments, at least one of the at least one detectors is a photodetector. In some embodiments, the capsule or spherocylinder comprises a cross-sectional diameter that is shorter than 10 cm, 9 cm, 8 cm, 7 cm, 6 cm, 5 cm, 4 cm, 3 cm, 2 cm, or 1 cm. In some embodiments, the semi-permeable membrane is a polyethersulfone membrane filter.

In some embodiments, at least one of the at least one biosensor component is an enzymatic biosensor or a non-enzymatic biosensor. In some embodiments, the non-enzymatic biosensor comprises an antibody, a binding protein, or a nucleic acid. In some embodiments, the enzymatic biosensor or non-enzymatic biosensor is a cellular biosensor comprising at least one microorganism. In some embodiments, the ingestible device further comprises at least one control component comprising a reference microorganism for calibration to remove background light and temperature induced drift. In some embodiments, the at least one microorganism is present in the device in a dormant state. In some embodiments, the at least one microorganism is combined with additional substances to aid in removing the at least one microorganism from its dormant state, to provide nutrients to the at least one microorganism, and/or to prolong the lifetime of the at least one microorganism. In some embodiments, at least one of the at least one microorganism comprises an engineered genetic circuit. In some embodiments, the output of the engineered genetic circuit is luminescence, fluorescence, ion flow, or turbidity.

In some embodiments, at least one of the at least one signal analyte is selected from the group consisting of a microorganism, a biomolecule, or an inorganic molecule. In some embodiments, at least one of the at least one signal analyte is a biomolecule. In some embodiments, the biomolecule is selected from the group consisting of henie, thiosulfate, and acyl-homoserine lactone.

In other aspects, the disclosure relates to methods of monitoring the health of a patient using an ingestible device as described above. In some embodiments, the method comprises orally administering the device to a patient and comparing the output of the device to a control. In some embodiments, the control is established through analysis of a population of healthy patients. In some embodiments, the patient is a human patient. In some embodiments, human patient is predisposed to a disease, disorder, morbidity, sickness, or illness. In some embodiments, the human patient has been diagnosed with a disease, disorder, morbidity, sickness, or illness.

These and other aspects of the invention are further described below.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1C. Probiotic E. coli can be engineered to sense blood in vitro and in vivo. FIG. 1A, Schematic of the blood sensor gene circuit. Extracellular heme is internalized through the outer membrane transporter ChuA and interacts with the transcriptional repressor HtrR to allow for transcription of the bacterial luciferase operon luxCDABE. FIG. 1B. Dose-response curves of prototype (V1) and optimized (V2) heme sensing genetic circuits in laboratory (MG1655) and probiotic (Nissle) strains of E. coli. Error bars represent SEM of three independent biological replicates. FIG. 1C, C57BL6/J mice were administered vehicle (PBS) or indomethacin (10 mg/kg) to induce gastrointestinal bleeding and inoculated with blood sensor E. coli Nissle cells the following day. Normalized luminescence values of fecal pellets were significantly higher in mice administered indomethacin compared to control animals (*P=0.04; Student's t-test; n=10).

FIGS. 2A-2E. Design and in vitro evaluation of MBED for miniaturized wireless sensing with cellular biosensors. FIG. 2A. Cross section, electrical system diagram, and front and back-side photos of the device. FIG. 2B. System photocurrent response measured without cells. The incident photon flux was supplied by green LED (λ=525 nm) and calibrated with an optical power meter (n=3 devices). FIG. 2C. Kinetic response of blood sensor MBED in bacterial growth media supplemented with 0 ppm and 500 ppm blood. FIG. 2D. Dose-response of blood sensor MBEDs in bacterial growth media containing different blood concentrations 2 h post-exposure. The left-most data point represents the background response in the absence of blood. FIG. 2E. MBEDs are a modular platform for detection of multiple gut-relevant small molecules by employing alternative probiotic biosensors. HrtR-, LuxR- and ThsRS-containing E. coli Nissle strains in MBEDs were exposed to 500 ppm blood, 100 nM acyl-homoserine lactone (AHL) or 10 mM thiosulfate for 2 h. In C-E, error bars denote the SEM for 3 independent biological replicates conducted with different MBEDs. *P<0.05, **P<0.01, Student's t test.

FIGS. 3A-3E. MBEDs can rapidly detect porcine gastric bleeding. FIG. 3A. Schematic depicting experiment flow which consisted of blood administration in neutralization solution, capsule deposition, and wireless readout to commercial receiver connected to a laptop or a cellular phone. FIG. 3B. Endoscopic image of a device immersed in gastric contents. FIG. 3C. X-ray image of a device positioned inside the stomach. FIG. 3D. MBEDs deposited in gastric cavity can rapidly discriminate between pigs administered blood versus buffer control. Error bars denote SEM for six MBED experiments (3 animals on different days, 2 capsules per animal). FIG. 3E. Receiver operating characteristic (ROC) curve of MBED sensing over time. Perfect detection is achieved at t=120 minutes. *P<0.05, Student's t test.

FIG. 4. Capsule for sensing biomarkers in vivo with whole-cell bacterial sensors and wireless electronic readout.

FIGS. 5A-5D. Design and in vitro evaluation of prototype heme sensing genetic circuit. FIG. 5A. Promoter design of heme-responsive promoter. The TetR operator sites of a synthetic promoter based on the late promoter of bacteriophage lambda (P_{L(TetO)}) (Lutz R. and Bujard H., Nucleic Acids Res. 1997 Mar. 15; 25(6): 1203-10) were replaced with the operator DNA sequences to which HrtR binds. Spacing between the −10 and −35 sites was preserved. FIGS. 5B-5D. Dose-response curves of prototype genetic circuits in E. coli MG1655 in various concentrations of hemin (FIG. 5B), whole horse blood (FIG. 5C), and blood lysed in simulated gastric fluid (FIG. 5D). The genetic circuit contains P_{L(HrtO)}-luxCDABE alone (Lux), P_{L(HrtO)}-luxCDABE with the HrtR transcriptional repressor (HrtR+Lux), or P_{L(HrtO)}-luxCDABE, HrtR and the ChuA heme transporter (ChuA+HrtR+Lux). Luminescence values are measured 2 hours post-exposure to inducer and normalized to the optical density of the culture. Error bars represent SEM of three independent biological replicates.

FIGS. 6A-6D. Genetic circuit optimization by varying translational initiation strength of HrtR. FIGS. 6A-6C. Dose-response curves of heme-sensing genetic circuits in E. coli MG1655 in various concentrations of hemin (FIG. 6A), whole horse blood (FIG. 6B), and blood lysed in simulated gastric fluid (FIG. 6C). The translational initiation strength of HrtR was varied using different computationally-designed ribosome binding sites (RBS) (Salis H M, Methods Enzymol. 2011; 498: 19-42). FIG. 6D. Predicted RBS strengths. Luminescence values are measured 2 hours post-exposure to inducer and normalized to the optical density of the culture. Error bars represent SEM of three independent biological replicates.

FIG. 7. Blood biosensors responds to blood of different mammalian origins. E. coli Nissle blood sensor strains (Nissle V2 from FIG. 1B) were treated with various concentrations of human or horse blood lysed in simulated gastric fluid. Luminescence values are measured 2 hours post-exposure to inducer and normalized to the optical density of the culture. Error bars represent SEM of three independent biological replicates.

FIG. 8. Kinetic response of blood biosensor strain. E. coli Nissle blood biosensors (Nissle V2 from FIG. 1B) were treated with 10 μM hemin (brown), 1000 ppm blood (red) or PBS (black) and luminescence response was measured in a plate reader every 5 minutes for 2 hours. Luminescence values are normalized to the optical density of the bacterial culture, Error bars represent SEM of three independent biological experiments.

FIG. 9. Transit time of E. coli Nissle 1917 through the murine gastrointestinal tract. C57BL/6J mice were inoculated with approximately 2×10⁸ CFU of blood biosensors by oral gavage (n=4). Fecal pellets were collected from mice prior to gavage and at 2, 4, 6, 8 and 24 hours post-gavage and plated to determine CFU counts. All mice contained biosensor bacteria in their stool 6 h post-gavage and no colonization was observed. Dotted line indicates the limit of detection (LOD) of the assay.

FIGS. 10A-10B. Heme biosensors can detect blood in an in vivo murine model of indomethacin-induced gastrointestinal bleeding. FIG. 10A. Mice were inoculated with approximately 2×10⁸ CFU of E. coli Nissle blood sensors 6 hours prior to (Day 0) or 16 hours after (Day 1) administration of indomethacin (10 mg/kg) or PBS buffer as a negative control. Induction of bleeding was confirmed by guaiac test. Fecal pellets were collected from animals 6 hours post-gavage, homogenized and analyzed for luminescence production as well as plated to enumerate colony forming units (CFU). Luminescence values were normalized to cell number in fecal pellets. (n=10). *P<0.05, Student's t test. FIG. 10B. CFU counts in fecal pellets 6 hours post-gavage.

FIGS. 11A-11C. Capsule readout variation was characterized across optical input power, temperature change and fluid submersion. FIG. 11A. The coefficient of variation between measurements on three channels within a single device, characterized across input light intensity (N=3 devices). At low signal levels, the measurement standard deviation is limited by white noise (13%ᵣₘₛ noise at 1.3 pA). At higher signal levels, it is limited by mismatch between the channels (<6%ᵣₘₛ above 3p A). FIG. 11B. Residual variation induced by temperature change, post-calibration. The temperature was stepped from 35° C. to 40° C. (temperature change 5° C.) and the standard deviation across three sensor channels was measured (N=3 devices). FIG. 11C. Stability of the measurements from MBED devices in Simulated Gastric Fluid (SGF) for 72 h (n=3). For two devices, current values were stable for the duration of measurement. The third system operated for 36 h before corruption by humidity became evident.

FIGS. 12A-12H. Technical replicates of blood sensor MBED across various blood concentrations. Overnight cultures of E. coli Nissle blood biosensors were diluted in fresh 2×YTPG and loaded in an MBED in triplicates. Wild-type Nissle was loaded in the reference channel. The assembled device was submerged in pre-warmed LB supplemented with the indicated concentration of blood. Each line depicts a biological replicate of the mean response of a single MBED for a given concentration of blood. Error bars represent the standard deviation of the three replicate channels within a single device. FIG. 12A: 1000 ppm; FIG. 12B: 500 ppm; FIG. 12C: 250 ppm; FIG. 12D: 125 ppm; FIG. 12E: 62.5 ppm; FIG. 12F: 31.25 ppm; FIG. 12G: 15.625 ppm; and FIG. 12H: 0 ppm.

FIGS. 13A-13D. Design and characterization of acyl-homoserine lactone (AHL) and thiosulfate-responsive biosensors. FIG. 13A. AHL binds to the transcriptional activator LuxR that activates transcription of the luxCDABE operon downstream of the Pₗᵤₓ promoter. FIG. 13B. Titrating increasing amounts of AHL yields higher levels of luminescence. FIG. 13C. The ThsRS two-component system mediated thiosulfate-inducible expression of the luxCDABE operon from the P_{phsA} promoter. Thiosulfate binds to the membrane bound ThsS histidine kinase that, in turn, phosphorylates the ThsR response regulator such that it can activate transcription from P_{phsA}. FIG. 13D. Titrating increasing amounts of ThsS yields higher levels of luminescence. Error bars indicate SEM from three independent biological replicates.

FIGS. 14A-14B. Mobile phone and 900 MHz wireless receiver dangle used for visualizing MBED measurement results and logging them to the cloud. The receiver dongle connects to the phone via USB and delivers packets received wirelessly from the MBED device to application software. The software uploads data to a cloud service and performs visualization for the user. Displayed are views of the front (FIG. 14A) and the back (FIG. 14B) of the mobile phone.

FIGS. 15A-15B. Application software displaying MBED measurement results to the user on a mobile phone. Representative data received from the MBED device during a porcine study with administration of (FIG. 15A) the buffer solution, and (FIG. 15B) the blood solution.

FIG. 16. Individual replicates of blood sensing MBEDs in the pig gastric environment. Blood sensor MBEDs were deposited in the gastric cavity of pigs administered neutralization solution containing 0.25 mL of blood (red) or buffer alone (black). Readings from MBEDs were wirelessly collected for 120 minutes following device deposition. Dark trace represent the mean of 6 replicate MBEDs (3 animals on different days, 2 devices per pig) and pale traces indicate the individual current values for a given MBED.

FIG. 17. Functional blood biosensing genetic circuits are necessary for MBED detection of blood in the pig gastric environment. E. coli Nissle strains containing a functional biosensor circuit (Sensor), a circuit lacking the luciferase output (Δlux) and a circuit lacking the heme transporter ChuA (ΔchuA) were loaded into a MBED. Devices were deposited in the stomach of animals administered neutralization solution spiked with blood or with buffer alone. MBED readings were wirelessly collected for 120 minutes post-device deposition. Only channels that correspond to functional biosensors in pigs administered blood display high levels of luminescence. Endogenous levels of heme in the pig stomach as well as the cellular response to the pig gastric environment are not sufficient to generate high levels of bioluminescence. Error bars denote SEM for six MBED experiments (3 animals on different clays, 2 capsules per animal). Graph plots proceeding from top to bottom at 120 min: + Blood, Senor; − Blood, Sensor; − Blood, Δlux; + Blood, Δlux and − Blood, ΔchuA; + Blood, ΔchuA.

FIG. 18 shows a block diagram of the electrical component of an MBED, such as the MBED of FIG. 2A, according to an illustrative embodiment.

### DETAILED DESCRIPTION

The scaling of semiconductor microelectronics over the past few decades has delivered sophisticated, highly sophisticated platforms for sensing, computating, and wireless communication (Otis B. and Parviz B., Google Off. Blog, 2014; Wang H., IEEE Microw. Mag., Jill 2013; 14(5): 110-30; Norian H., et al., Lab Chip., 2014 Oct. 21; 14(20): 4076-84). These platforms have been incorporated into devices that monitor health and disease. For example, in the gastrointestinal tract, electronic capsules have been deployed for taking visual images (Iddan G., et al., Nature, 2000 May; 405(6785): 417) (15), delivering drugs while measuring temperature and pH (van der Schaar P. J., et al., Gastrointest. Endosc., 2013 September; 78(3): 520-28), and recording patient compliance (Hafezi H., et al., IEEE Trans. Biomed. Eng., 2015 January; 62(1): 99-109). While electronics provide a versatile interface for collecting, processing, and sharing information, their ability to directly sense biomolecules in vivo has been limited due to their dependence on labile biochemical transducers that necessitate large, power-demanding circuits for sensitive detection.

By combining the environmental resilience and natural sensing properties of bacterial cells with the complex data processing and wireless transmission afforded by microelectronics, a device capable of in vivo biosensing in harsh, difficult-to-access environments was developed. Using gastrointestinal bleeding as a proof-of-concept model system, strategies for genetic circuit design and optimization, fabrication of an ingestible low-power, wireless luminometer, and validation of integrated system functionality were demonstrate both in vitro and in a large animal model.

As the field of whole-cell biosensors matures, newly developed sensors of clinically-relevant biomarkers can be rapidly integrated into a MicroBioElectronic Device (MBED) to perform minimally-invasive detection in the gastrointestinal tract. By creating a larger array of photodetectors, a panel of biochemical tests can be simultaneously performed by a single device. With a test panel of candidate biomolecules, MBEDs enable studies of biochemical activity in anatomical regions that are traditionally difficult to access and lead to the discovery of novel clinical biomarkers associated with health or disease. Further integration of electronic modules, such as photodetectors, microprocessor and transmitter, in a single integrated circuit allows for further miniaturization of MBEDs as well as lower power consumption. Additional measurement channels also enables more precise biochemical readings, as the response of replicate biosensors within the same device could be averaged to mitigate the inherent variance of biological sensors as well as the heterogeneity of the complex gastrointestinal environment. This integration of biological engineering and semiconductor electronics offers opportunities to transform diagnosis, management, and monitoring of health and disease.

Disclosed herein are novel devices comprising small, ultra-low power microelectronic components that overcome these limitations. For example, integration of electronic modules, such as photodetectors, microprocessor and transmitter, in a single integrated circuit can allow for further miniaturization of MBEDs as well as lower power consumption.

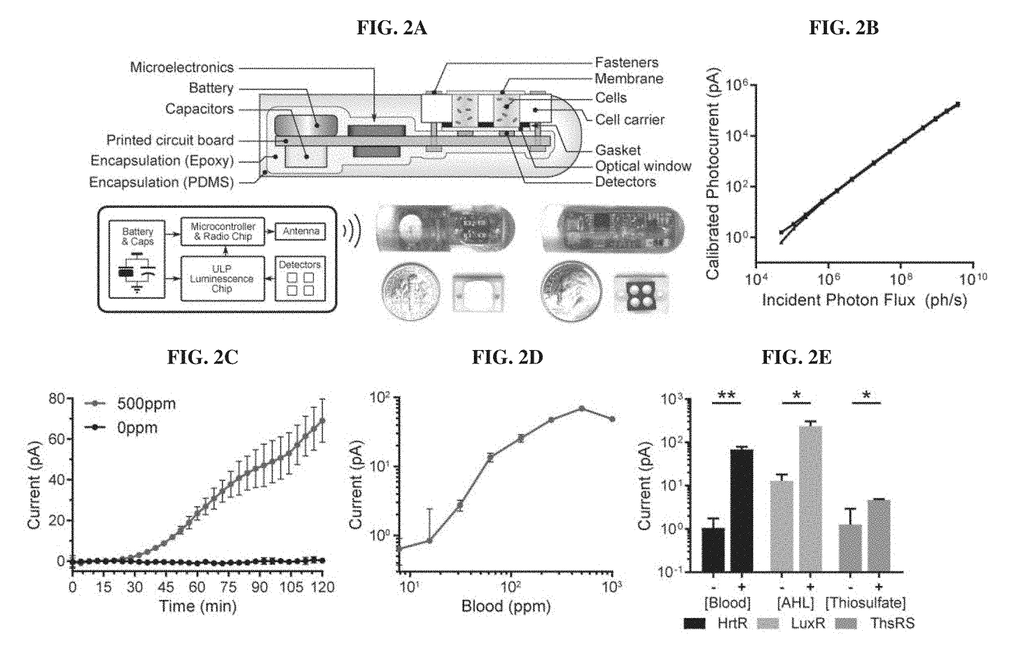

FIG. 2A illustrates a cross section, electrical system diagram, and front and back-side photos of an MBED for miniaturized wireless sensing with cellular biosensors. The device includes multiple detectors, such as photodetectors including NPN photodetector transistors. Each detector may be associated with a measurement channel, and all or a portion of the detectors may detect signals indicating an output of the engineered genetic circuit. For example, a genetic circuit may be configured to output luminescence in response to the presence of an analyte. In some embodiments, a control detector may detect background luminescence and/or other sources of common mode signals.

The detectors are connected to an ultra-low power (ULP) luminescence chip, which may be configured to determine when the detectors are indicating the presence of an analyte. For example, the ULP luminescence chip may measure voltage and/or current signals generated by photodetectors in response to luminescence from an engineered genetic circuit. The ULP luminescence chip may include any suitable circuitry for interfacing with the detectors and receiving signals indicating the presence of an analyte. For example, the detectors may be used to charge a capacitance, and the ULP luminescence chip may measure the voltage across the capacitance. In some embodiments, the output level of an engineered genetic circuit may be determined based on the amount of time that is required for the respective detector to charge the capacitance, the amount of time being related to a current signal generated by the detector in response to the output luminescence) of the engineered genetic circuit.

The ULP luminescence chip interfaces with a microcontroller and radio chip that may be used to wirelessly transmit indications of the detector outputs to a receiver. The wireless transmission allows for monitoring that may substantially continuous and performed in real time. For example, data may be transmitted at regular intervals or in response to signals from the detectors. In some embodiments, as shown in FIG. 2A, the electrical component may utilize a power source including both a battery and a capacitor, which may provide power at a relatively high rate needed for wireless transmissions. In some embodiments, since the power required to transmit data is much larger than the power required for detecting an analyte, the transmitter may be configured to transmit only after certain intervals have passed. In further embodiments, the transmitter may transmit data only once signals from all or a portion of the detectors exceeds a reference signal. For example, the ULP luminescence chip may count a number of oscillator cycles needed to charge the capacitances associated with each detector beyond a reference voltage, and the radio chip may only transmit the counted numbers of cycles when a threshold number of the capacitances are charged beyond the reference voltage. This allows the device to save power without adversely impacting the monitoring.

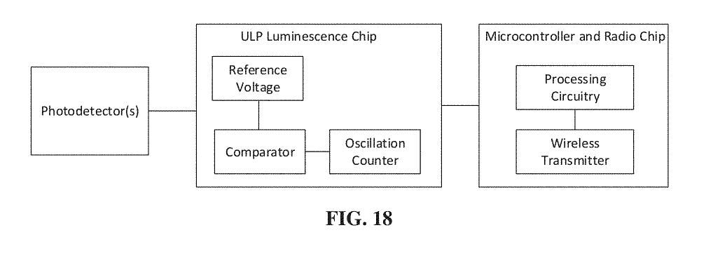

FIG. 18 shows a block diagram of the electrical component of an MBED, such as the MBED of FIG. 2A, according to an illustrative embodiment. It should be appreciated that the component layouts shown are provided by way of illustration and other sufficiently miniaturized circuits may be employed without departing from the scope of the present application.

The electrical component includes at least one photodetector configured to charge a capacitance. In some embodiments, the capacitance is internal to the photodetector. The photodetectors may be associated with at least one biosensor component of the MBED. One or more photodetectors may be used as controls to detect common mode signals that may be subsequently suppressed. The photodetectors may provide respective voltage signals, indicating the charge stored by the capacitance, to a comparator that may be configured to compare the respective voltage signals to a reference voltage. When the voltage signal from one of the photodetectors exceeds the reference voltage, an oscillation counter may store a number of oscillator cycles that occurred during the time required for the photodetector to charge the capacitance. When the voltage signals from all or a portion of the photodetectors exceed the reference voltage, the wireless transmitter may wirelessly transmit the numbers of oscillator cycles stored for each of the photodetectors with voltages that exceeded the threshold.

In some embodiments, the device contains a calibration scheme for detecting and removing background light and temperature-induced drift (see e.g., Material and Methods).

The electrical component of the device can be made small enough to perform detection in space-constrained environments. The low power consumption of the device, which in some embodiments is on the order of 10 uW or less, enables the use of a millimeter-scale battery for extended measurement. For example, in some embodiments, the device comprises a battery, wherein the longest cross-sectional measurement of the battery is shorter than 10 mm, 9 mm, 8 mm, 7 mm, 6 mm, 5 mm, 4 mm, 3 mm, 2 mm, or 1 mm. Other power sources known to those of skill in the art can be utilized in the device, in addition to or in place of the battery, such as energy harvesting component(s) or wireless power transfer component(s).

Semiconductor integration and packaging allow all components of the device to be placed in a compact arrangement. For example, in some embodiments, the device is encapsulated within a capsule or spherocylinder comprising a cross-sectional diameter that is shorter than 100 cm, 50 cm, 25 cm, 20 cm, 15 cm, 10 cm, 9 cm, 8 cm, 7 cm, 6 cm, 5 cm, 4 cm, 3 cm, 2 cm, 1 cm, 0.9 cm, 0.8 cm, 0.7 cm, 0.6 cm, 0.5 cm, 0.4 cm, 0.3 cm, 0.2 cm, or 0.1 cm. In some embodiments, the device is ingestible (or “suitable for ingestion”) or implantable.

The devices described herein are capable of detecting a wide range of analytes or combinations of analytes. In some embodiments, an analyte is selected from the group consisting of a microorganism, a biomolecule, or an inorganic molecule. As used herein, the term “biomolecule” refers to a molecule generated by an organism. In some embodiments, the biomolecule is a macromolecule. Examples of macromolecules include, but are not limited to, proteins (i.e., polypeptides), carbohydrates, lipids, nucleic acids (i.e., polynucleic acids), and combinations thereof. In some embodiments, the biomolecule is a small molecule such as a metabolite, secondary metabolite, or a natural product. Examples of small molecule biomolecules are known to those having ordinary skill in the art In some embodiments, the biomolecule is selected from the group consisting of heme, thiosulfate, and acyl-homoserine lactone. As used herein, the term “inorganic molecule” refers to any molecule (including an element) that is not a biomolecule. In some embodiments, the inorganic molecule is a gas, a heavy metal (e.g., Hg, Cd, Ni, Co, Zn, Cu, Pb, Au), a PCB, or a pesticide.

In some embodiments, the device facilitates the detection of numerous analytes. For example, by creating a large array of photodetectors, a panel of biochemical tests can be simultaneously performed by a single device.

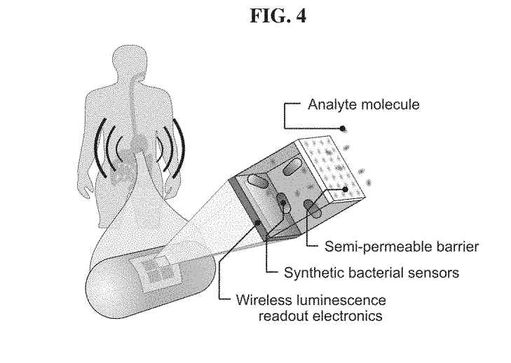

Also described herein are MBEDs that combine biosensors with the ultra-low power electronics described above to enable in situ detection of analytes (FIG. 4). As such in some embodiments, a device comprises an electronic component as described above and a biosensor component. Various examples of biosensors are known to those having skill in the art (Lim H. G., et al., Curr. Opin. Biotechnol, 2018 Feb. 3; 54: 18-25; Ragavan K. V., et al., Biosens. Bioelectron. 2018 May 15; 105: 188-210; Ali J., et al., J. Biosens. Biolectron., 2017; 8(1): doi: 10.4172/2155-6210.1000235, Justino C. I. L., et al., Sensors (Basel), 2017 Dec. 15; 17(12): pii: E2918; Huang Y., et al., Sensors (Basel), 2017 Oct. 17; 17(10): pii: E2375), the contents of which are incorporated herein.

In some embodiments, the biosensor component is sensitive to the presence of at least one signal analyte and communicates the presence of the at least one signal analyte to the electronic component. As used herein the term “sensitive to the presence of” refers to the ability of a biosensor to detect the presence of an analyte above a threshold amount. As such, the sensitivity of a biosensor will vary. Methods of determining the sensitivity of a particular biosensor are known to those having skill in the art (see e.g., Example 1).

As used herein the term “communicates the presence of” refers to the generation of an output that can be sensed by the electronic component of the device. In some embodiments, the output of the engineered genetic circuit is luminescence chemiluminescence, triboluminescence, photoluminescence, fluorescence, phosphorescence), ion flow (e.g., resulting from the opening of a channel or a redox reaction), or turbidity (e.g., cell growth that precludes the passage of light). For example, the sensing of a target analyte by a biosensor may generate light, which can be detected by photodetectors embedded in the electronic component. These electrical signals can then be processed by integrated bioluminescence detection incorporated into the circuit (Nadeau P., et al., IEEE, 2017 Mar. 6; doi10.1109/ISSCC.2017.7870406) and transmitted wirelessly from the device to an external radio or cellular phone for convenient readout.

In some embodiments, the communication is proportional to the abundance of the at least one signal analyte (i.e., the strength of a signal increase as the abundance of the analyte increases).

In some embodiments, the biosensor lies adjacent to readout electronics, separated from the outside environment by a semi-permeable membrane that permits diffusion of analytes. As used herein, the term “permits diffusion” relates to the pore size of the semi-permeable membrane. If a barrier permits the diffusion of an analyte, the radius of the pore of the membrane is larger than the radius of the analyte (e.g., Stokes radius). In some embodiments, the semi-permeable membrane is a polyethersulfone (PES) membrane filter.

In some embodiments, at least one of the at least one biosensor is an enzymatic biosensor or a non-enzymatic biosensor. An enzymatic biosensor, as used herein, comprises an enzyme that recognizes the target analyte to produce an output that can be sensed by the electronic component of the device. The output may be a signal generated through: 1) the enzymatic conversion of the analyte into a new product; 2) analyte-mediated inhibition or activation of the enzyme; or 3) analyte-mediate modification of enzyme properties. As used herein, the term “enzyme” refers to a biomolecule that acts as a catalyst to bring about a specific biochemical reaction.

In contrast, a non-enzymatic biosensor does not require interaction between an enzyme and a target analyte. For example, in some embodiments, a non-enzymatic biosensor comprises a protein channel that facilitates the signal flow (or output) when in the presence of an analyte. In some embodiments, a non-enzymatic biosensor comprises an antibody or a binding protein that recognizes the presence of an analyte. In some embodiments, the non-enzymatic biosensor comprises a nucleic acid that hybridizes to an analyte or otherwise hinds to it (e.g., as an aptamer). In some embodiments, the non-enzymatic biosensor comprises of a transcription factor that alters gene expression upon binding to an analyte.

In some embodiments, the enzymatic biosensor or non-enzymatic biosensor is a cellular biosensor comprising at least one microorganism. As used herein, the term “microorganism” refers to microscopic living organisms including archaea, bacteria, fungi, protista, microbial mergers or symbionts, planarians (e.g., C. elegans), and suspensions of mammalian cells, plant cells, or insect cells. In some embodiments, the cellular biosensor is an E. coli bacterium. In some embodiments, the at least one microorganism is present in the device in a dormant state. For example, in some embodiments the at least one microorganism is freeze-dried or lyophilized prior to or during device manufacture. Microorganisms present in the device in a dormant state may be removed from the dormant state prior to device use (e.g., through hydration) or as a result of device use. In some embodiments, the at least one microorganism is combined with additional substances to aid in removing the at least one microorganism from its dormant state (e.g., a wetting agent), to provide nutrients to the at least one microorganism, and/or to prolong the lifetime of the at least one microorganism in environments sub-optimal for the at least one microorganism (e.g., low pH or high pH).

Microorganisms living on and in the human body constantly interrogate their biochemical surroundings and alter gene expression to adapt to changing environments. Whole-cell biosensors harness this sensing ability to detect analytes of interest. In some embodiments, the cellular biosensor lies adjacent to readout electronics in individual wells separated from the outside environment by a semi-permeable membrane that confines cells in the device and allows for diffusion of analytes.

Synthetic biology enables the robust engineering of living cells with increasingly complex genetic circuits to sense multiple biological inputs and control gene expression (Brophy J. A. and Voigt C. A., Nat. Methods., 2014 May; 11(5): 508-20.). In some embodiments, the cellular biosensor comprises an engineered genetic circuit. Examples of engineered genetic circuits are provided in Example 1, Example 2, and Example 5. Other non-limiting examples of engineered genetic circuits for detection of analytes of interest include: US 2017/0058282 (describing genetically engineered sensors for in vivo detection of bleeding), US 2017/0360850 (describing genetically engineered sensors for in vivo detection of hydrogen peroxide, nitric oxide, inflammatory cytokines such as IL-6, IL-18, or TNF-alpha), US 2017/0335411 (describing genetically engineered sensors for in vivo detection of signals including chemical signals), and US 2017/0255857 (describing genetically engineered analog-to-digital biological converter switches and their use in biological systems including as sensors).

In some aspects, the disclosure relates to methods of detecting at least one signal analyte. In some embodiments, the method comprises contacting a device as described above with a sample and comparing the output of the device to a control, wherein the control contains a known quantity of the at least one signal analyte. As described herein, the term “lacks a detectable quantity” relates to a threshold amount of analyte that is detectable by a device above background level. As such, the term “lack a detectable quantity” is tied to the sensitivity of the particular device. Methods of determining the sensitivity of a particular device are known to those having skill in the art (see e.g., Materials and Methods and Example 5).

Whole-cell biosensors have been used previously to detect analytes associated with environmental contamination (Roggo C., and van der Meer J. R., Curr. Opin. Biotechnol. 2017 June; 45: 24-33). In some embodiments, the sample is selected from the group consisting of soil, water, air, or food.

The integration of biological engineering and semiconductor electronics offers opportunities to transform diagnosis, management, and monitoring of health and disease. Previously described biosensors have been developed to sense clinically relevant biomarkers in serum or urine ex vivo (Courbet A., et al., Sci. Transl. Med., 2015 May 27; 7(289): 289-83) as well as gut biomolecules supplemented in diet (Kotula J. W., et al., Proc. Natl. Acad. Sci. U.S.A, 2014 Apr. 1; 111(13): 4838-43; Mimee M., et al., Cell Syst., 2016 March 23; 2(3): 214; Lim B., et al., Cell, 2017 Apr. 20; 169(3): 547-58.e15) or generated during disease (Daeffler K. N., et al., Mol. Syst. Biol., 2017 Apr. 3; 13(4): 923; Riglar D. T., et al., Nat. Biotechnol., 2017 July; 35(7): 653-58; Pickard J. M., et al., Nature, 2014 Oct. 30; 514(7524): 638-41). However, despite their promise as non-invasive diagnostics, previously described biosensors have yet to be employed for clinically compatible testing in an unobtrusive, real-time, and user-friendly way. Current research applications of ingestible biosensors in animal models rely on cumbersome analysis of bacterial gene expression or DNA in stool samples (Kotula J. W., et al., Proc. Natl. Acad. Sci. U.S.A, 2014 Apr. 1; 111(13): 4838-43; Mimee M., et al., Cell Syst., 2016 Mar. 23; 2(3): 214; Lim B., et al., Cell, 2017 Apr. 20; 169(3): 547-58.e15; Daeffler et al., Mol. Syst. Biol., 2017 Apr. 3; 13(4): 923; Riglar D. T., et al., Nat. Biotechnol., 2017 July; 35(7): 653-58; Pickard J. M., et al., Nature, 2014 Oct. 30; 514(7524): 638-41), rather than real-time reporting from within the body. Moreover, biomolecular monitoring is often impeded by access to the remote and complex environments. The MicroBioElectronic Devices (MBEDs) described herein overcome the limitation of the prior art and are capable of in vivo biosensing in harsh, difficult-to-access environments.

In some aspects, the disclosure relates to methods of monitoring the health of a patient. In some embodiments, the method comprises contacting a device as described above with a patient and comparing the output of the device to a control, wherein the control is a reference value that optionally is established through analysis of a population of healthy patients.

In some embodiments the patient is a domestic or wild animal. In some embodiments, the patient is a human patient.

In some embodiments, the contacting occurs by oral administration of the device to the patient or other delivery methods that result in deposition of the device into the esophagus, stomach, or intestine. In some embodiments, deposition arises through the consuming or the swallowing of the device by the patient. In other embodiments, the contacting of the device with the patient occurs by implantation, such as by surgical implantation. In some embodiments, the contacting occurs by attachment to the surface of the patient, e.g., the skin.

In some embodiments, the patient is being monitored in a pre-clinical or clinical trial.

In some embodiments, the patient is a human patient. In some embodiments, the human patient is predisposed to a disease, disorder, morbidity, sickness, or illness. In some embodiments, the human patient has been diagnosed with a disease, disorder, morbidity, sickness, or illness.

### Examples

### Materials and Methods

Bacterial Strains and Culture Conditions:

Routine cloning and plasmid propagation was performed in E. coli DH5a, Gene circuits were initially prototyped in E. coli MG1.655 and were transferred into probiotic E. coli Nissle 1917 for capsule and in vivo experiments. Cells were routinely cultured at 37° C. in Luria-Bertani (LB) media (Difco). Where appropriate, growth media was supplemented with antibiotics at the following concentrations: 30 μg/mL kanamycin, 100 μg/mL carbenicillin, 25 μg/mL chloramphenicol and 100 μg/mL spectinomycin.

Genetic Part and Plasmid Construction:

Genetics parts and plasmids used in this study are listed in TABLE 1 and TABLE 2 and will be available from Addgene upon publication. All plasmids were constructed by combining PCR fragments generated by Kapa Hifi Polymerase using Gibson Assembly (Gibson D. G., et al., Nat Meth., 2009 May; 6(5): 343-45). Assembly products were transformed into chemically competent E. coli DH5a (Chung C. J., et al., Proc. Natl. Acad. Sci. U.S.A, 1989 April; 86(7): 2172-75) and sequences were confirmed using Sanger sequencing. Ribosome binding sites (RBSs) of variable strengths were computationally designed using the Salis lab RBS calculator (Espah Borujeni A., et al., Nucleic Acids Res., 2014 February; 42(4): 2646-59; Salis H. M., et al., Nat. Biotechnol., 2009 October; 27(10): 946-50),

TABLE 1
Genetic Parts

| | SEQ | | |
|---|---|---|---|
| Part | ID | | |
| Name | NO: | Type | DNA sequence |
| HrtRO | 1 | HrtR operator | ATGACACAGTGTCAT |
| | | sequence | |
| PL(HrtO) | 2 | Heme-inducible | ATAAATGACACAGTGTCATTTGACAAAATGACACAGTG |
| | | Promoter | TCATGATACTGAGCACA |
| Plux | 3 | AHL-inducible | ACCTGTAGGATCGTACAGGTTTACGCAAGAAAATGGTT |
| | | promoter | TGTTATAGTCGAATAAA |
| PphsA | 4 | Thiosulfate- | TTCAAGCATTATTATGCTGTTTTTTGAAGTGAATGTGCG |
| | | inducible promoter | GCCATCTAGCCGCACATTTTGCATCTAAAACATGCAGT |
| | | | CATCAGCAAAATAATAAACTTTTCCCCAATATGTGGTTT |
| | | | ACCACAATTTACAGGAATTCACTCCTGTGGTGGTGCAA |
| | | | ATTTGAACTGTGAATTGCTTCACAAACGCCGCTATCGC |
| | | | AATGTCAGTATGTGGTTTACCACAATATCTAATATCACT |
| | | | CTGCTCAATAACAATGATGAAAACCTTAGGAAGAAGTT |
| | | | AATTGTGTTAAACAGTTAACTAGGGGCTTTATCTAACG |
| | | | CTCTCCTAAGGACAACTGTCATTGGGAGATTTAAC |
| J23107 | 5 | Constitutive | TTTACGGCTAGCTCAGCCCTAGGTATTATGCTAGCACAT |
| | | Promoter + RBS for | TTCCAACACTAACCCAAGGGAGCTTTAAATC |
| | | ChuA | |
| ProD | 6 | Consitutive | CACAGCTAACACCACGTCGTCCCTATCTGCTGCCCTAG |
| | | Promoter for HrtR | GTCTATGAGTGGTTGCTGGATAACTTTACGGGCATGCA |
| | | | TAAGGCTCGTATAATATATTCAGGGAGACCACAACGGT |
| | | | TTCCCTCTACAAATAATTTTGTTTAACTTT |
| K176009 | 7 | Consitutive | TTTACGGCTAGCTCAGTCCTAGGTATTATGCTAGCACTA |
| | | Promoter + RBS for | GAAAAGAGGAGAAAACTAGA |
| | | LuxR | |
| J23104 | 8 | Constitutive | TTGACAGCTAGCTCAGTCCTAGGTATTGTGCTAGCCTAG |
| | | Promoter + RBS for | TATCGATCTCCATAACTATCCTATAGATC |
| | | ThsS | |
| J23105 | 9 | Constitutive | TTTACGGCTAGCTCAGTCCTAGGTACTATGCTAGCAGA |
| | | Promoter + RBS for | AATATAAAGAACGATCTATTTATCCGCGTAC |
| | | ThsR | |
| RBS1 | 10 | HrtR RBS variant | GCTATAAGAAAACACCCTTTATAATCTAGGTTAAT |
| RBS2 | 11 | HrtR RBS variant | ATTAAAGAGGAGAAAG |
| RBS3 | 12 | HrtR RBS variant | TATACTCTAATTAATCACATAATAAGGACGAATTT |
| RBS4 | 13 | HrtR RBS variant | AGCCGCAAACATATAAGGAGGAACCCC |
| HrtR | 14 | Heme-responsive | ATGCCAAAATCAACCTATTTTAGTCTTTCTGACGAAAA |
| | | transcriptional | ACGAAAACGTGTCTATGATGCCTGTTTACTAGAATTTCA |
| | | repressor | AACGCACTCTTTCCATGAAGCTAAAATCATGCACATCG |
| | | | TAAAAGCACTTGATATCCCAAGAGGAAGTTTTTATCAA |
| | | | TACTTTGAAGATTTGAAGGATTCATACTATTATATCTTG |
| | | | TCACAGGAAACTGTCGAGATTCATGATTTATTTTTTAAT |
| | | | TTACTAAAAGAATATCCTCTAGAAGTTGCTCTTAATAA |
| | | | ATACAAGTATCTTCTTCTTGAAAATTTAGTAAATTCGCC |
| | | | CCAATATAATCTTTATAAATATCGATTTTTAGATTGGAC |
| | | | TTATGAATTAGAAAGAGATTGGAAGCCTAAAGGCGAG |
| | | | GTAACTGTTCCCGCTCGTGAACTTGATAATCCTATTTCC |
| | | | CAAGTATTAAAATCAGTCATTCACAATCTAGTTTATCGC |
| | | | ATGTTTAGTGAAAATTGGGATGAACAAAAGTTTATTGA |
| | | | AACTTACGATAAAGAAATCAAATTGCTCACAGAGGGCT |
| | | | TGCTTAATTATGTTACTGAAAGCAAAAAATAG |
| ChuA | 15 | Outer membrane | ATGTCACGTCCGCAATTTACCTCGTTGCGTTTGAGTTTA |
| | | heme transporter | TTGGCCTTAGCTGTTTCTGCCACCTTGCCAACGTTTGCT |
| | | | TTTGCTACTGAAACCATGACCGTTACGGCAACGGGGAA |
| | | | TGCCCGTAGTTCCTTCGAAGCGCCTATGATGGTCAGCGT |
| | | | CATCGACACTTCCGCTCCTGAAAATCAAACGGCTACTT |
| | | | CAGCCACCGATCTGCTGCGTCATGTTCCTGGAATTACTC |
| | | | TGGATGGTACCGGACGAACCAACGGTCAGGATGTAAAT |
| | | | ATGCGTGGCTATGATCATCGCGGCGTGCTGGTTCTTGTC |
| | | | GATGGTGTTCGTCAGGGAACGGATACCGGACACCTGAA |
| | | | TGGCACTTTTCTCGATCCGGCGCTGATCAAGCGTGTTGA |
| | | | GATTGTTCGTGGACCTTCAGCATTACTGTATGGCAGTGG |
| | | | CGCGCTGGGTGGAGTGATCTCCTACGATACGGTCGATG |
| | | | CAAAAGATTTATTGCAGGAAGGACAAAGCAGTGGTTTT |
| | | | CGTGTCTTTGGTACTGGCGGCACGGGGGACCATAGCCT |
| | | | GGGATTAGGCGCGAGCGCGTTTGGGCGAACTGAAAATC |
| | | | TGGATGGTATTGTGGCCTGGTCCAGTCGCGATCGGGGT |
| | | | GATTTACGCCAGAGCAATGGTGAAACCGCGCCGAATGA |
| | | | CGAGTCCATTAATAACATGCTGGCGAAAGGGACCTGGC |
| | | | AAATTGATTCAGCCCAGTCTCTGAGCGGTTTAGTGCGTT |
| | | | ACTACAACAACGACGCGCGTGAACCAAAAAATCCGCA |
| | | | GACCGTTGGGGCTTCTGAAAGCAGCAACCCGATGGTTG |
| | | | ATCGTTCAACAATTCAACGCGATGCGCAGCTTTCTTATA |
| | | | AACTCGCCCCGCAGGGCAACGACTGGTTAAATGCAGAT |
| | | | GCAAAAATTTTATTGGTCGGAAGTCCGTATTAATGCGCA |
| | | | AAACACGGGGAGTTCCGGCGAGTATCGTGAACAGATA |
| | | | ACAAAAGGAGCCAGGCTGGAGAACCGTTCCACTCTCTT |
| | | | TGCCGACAGTTTCGCTTCTCACTTACTGACATATGGCGG |
| | | | TGAGTATTATCGTCAGGAACAACATCCGGGCGGCGCGA |
| | | | CGACGGGCTTCCCGCAAGCAAAAATCGATTTTAGCTCC |
| | | | GGCTGGCTACAGGATGAGATCACCTTACGCGATCTGCC |
| | | | GATTACCCTGCTTGGCGGAACCCGCTATGACAGTTATC |
| | | | GCGGTAGCAGTGACGGTTACAAAGATGTTGATGCCGAC |
| | | | AAATGGTCATCTCGTGCGGGGATGACTATCAATCCGAC |
| | | | TAACTGGCTGATGTTATTTGGCTCATATGCCCAGGCATT |
| | | | CCGCGCCCCGACGATGGGCGAAATGTATAACGATTCTA |
| | | | AGCACTTCTCGATTGGTCGCTTCTATACCAACTATTGGG |
| | | | TGCCAAACCCGAACTTACGTCCGGAAACTAACGAAACT |
| | | | CAGGAGTACGGTTTTGGGCTGCGTTTTGATGACCTGAT |
| | | | GTTGTCCAATGATGCTCTGGAATTTAAAGCCAGCTACTT |
| | | | TGATACCAAAGCGAAGGATTACATCTCCACGACCGTCG |
| | | | ATTTCGCGGCGGCGACGACTATGTCGTATAACGTCCCG |
| | | | AACGCCAAAATCTGGGGCTGGGATGTGATGACGAAATA |
| | | | TACCACTGATCTGTTTAGCCTTGATGTGGCCTATAACCG |
| | | | TACCCGCGGCAAAGACACCGATACCGGCGAATACATCT |
| | | | CCAGCATTAACCCGGATACTGTTACCAGCACTCTGAAT |
| | | | ATTCCGATCGCTCACAGTGGCTTCTCTGTTGGGTGGGTT |
| | | | GGTACGTTTGCCGATCGCTCAACACATATCAGCAGCAG |
| | | | TTACAGCAAACAACCAGGCTATGGCGTGAATGATTTCT |
| | | | ACGTCAGTTATCAAGGACAACAGGCGCTCAAAGGTATG |
| | | | ACCACTACTTTGGTGTTGGGTAACGCTTTCGACAAAGA |
| | | | GTACTGGTCGCCGCAAGGCATCCCACAGGATGGTCGTA |
| | | | ACGGAAAAATTTTCGTGAGTTATCAATGGTAA |
| ThsS | 16 | Thiosulfate- | ATGTCCCGCCTGCTGCTGTGTATCTGTGTTCTGCTGTTC |
| | | responsive | TCTTCTGTGGCGTGGTCTAAACCGCAGCAGTTTTATGTG |
| | | histidine kinase | GGCGTACTGGCTAACTGGGGTCATCAGCAAGCCGTTGA |
| | | | ACGTTGGACCCCGATGATGGAGTATCTGAACGAACATG |
| | | | TGCCGGACGCGGAATTTCACGTCTACCCGGGCAACTTC |
| | | | AAAGCACTGAACCTGGCAATGGAACTGGGCCAGATTCA |
| | | | GTTCATTATCACTAACCCGGGCCAATATCTGTACCTGAG |
| | | | CAATCAGTACCCGCTGTCTTGGCTGGCGACCATGCGTTC |
| | | | TAAGCGTCACGATGGTACCACTTCTGCGATCGGTTCCG |
| | | | CCATTATTGTCCGCGCGGACAGCGACTACCGCACCCTG |
| | | | TACGACCTGAAAGGTAAAGTGGTGGCTGCGTCCGACCC |
| | | | GCATGCTCTGGGTGGCTACCAAGCGACCGTCGGTCTGA |
| | | | TGCATTCCCTGGGCATGGATCCGGACACCTTCTTCGGTG |
| | | | AAACCAAGTTTCTGGGCTTTCCACTGGATCCGCTGCTGT |
| | | | ACCAAGTTCGTGATGGCAACGTTGACGCGGCCATTACC |
| | | | CCACTGTGCACTCTGGAGGACATGGTTGCACGCGGCGT |
| | | | ACTGAAATCTTCCGATTTTCGTGTGCTGAACCCTAGCCG |
| | | | CCCGGATGGTGTAGAATGCCAGTGCTCTACCACCCTGT |
| | | | ACCCGAACTGGTCTTTCGCTGCGACTGAGTCTGTATCCA |
| | | | CCGAACTGTCTAAAGAAATCACGCAGGCACTGCTGGAA |
| | | | CTGCCATCCGACAGCCCGGCAGCTATCAAAGCGCAACT |
| | | | GACCGGCTGGACCAGCCCGATCTCCCAACTGGCGGTAA |
| | | | TCAAACTGTTCAAAGAGCTGCACGTAAAAACCCCGGAC |
| | | | TCTAGCCGTTGGGAAGCCGTTAAGAAGTGGCTGGAAGA |
| | | | AAACCGTCACTGGGGTATCCTGTCTGTTCTGGTGTTCAT |
| | | | CATTGCAACGCTGTATCACCTGTGGATTGAATACCGCTT |
| | | | CCACCAAAAAAGCTCTTCTCTGATCGAATCTGAACGTC |
| | | | AGCTGAAACAGCAAGCTGTTGCCCTGGAACGTCTGCAA |
| | | | TCTGCTAGCATCGTTGGTGAAATTGGTGCGGGTCTGGC |
| | | | CCACGAGATTAATCAGCCGATCGCTGCAATTACCTCTT |
| | | | ATTCTGAAGGTGGCATCATGCGCCTGCAAGGTAAAGAA |
| | | | CAGGCGGATACGGATAGCTGCATCGAACTGCTGGAAAA |
| | | | AATCCACAAACAGAGCACTCGCGCAGGCGAAGTGGTG |
| | | | CACCGCATCCGTGGTCTGCTGAAACGTCGTGAAGCGGT |
| | | | GATGGTAGATGTTAACATCCTGACCCTGGTGGAAGAAT |
| | | | CCATCAGCCTGCTGCGTCTGGAGCTGGCACGTCGCGAA |
| | | | ATCCAGATCAACACTCAGATCAAAGGTGAACCGTTCTT |
| | | | CATTACTGCCGACCGCGTTGGCCTGCTGCAAGTTCTGAT |
| | | | TAACCTGATCAAAAACTCCCTGGACGCGATCGCTGAAT |
| | | | CTGATAATGCCCGTTCTGGTAAAATCAACATCGAACTG |
| | | | GACTTTAAAGAGTACCAGGTAAACGTCTCCATCATCGA |
| | | | TAACGGTCCGGGCCTGGCGATGGATTCTGACACTCTGA |
| | | | TGGCTACGTTTTACACTACCAAAATGGATGGCCTGGGC |
| | | | CTGGGTCTGGCAATCTGCCGCGAAGTTATCAGCAACCA |
| | | | CGACGGCCACTTCCTGCTGTCCAACCGTGACGACGGCG |
| | | | TTCTGGGCTGTGTGGCAACCCTGAATCTGAAAAAACGC |
| | | | GGTTCTGAAGTGCCGATCGAAGTCTAA |
| ThsR | 17 | Thiosulfate- | ATGCAGCAGCAAATCAACGGCCCGGTCTACCTGGTGGA |
| | | responsive | TGATGATGAAGCCATTATCGACTCCATCGATTTTTTGAT |
| | | response regulator | GGAGGGCTACGGTTACAAACTGAACTCGTTTAACTGCG |
| | | | GCGATCGCTTTTTGGCAGAAGTCGATCTCACCCAGGCA |
| | | | GGATGTGTAATTCTGGATGCGCGTATGCCAGGCTTAAC |
| | | | TGGTCCTCAGGTGCAACAGCTGCTGAGCGACGCGAAAA |
| | | | GCCCGCTTGCGGTCATCTTCCTGACCGGCCATGGCGAT |
| | | | GTTCCGATGGCGGTTGATGCGTTCAAAAATGGCGCGTT |
| | | | CGATTTCTTTCAAAAACCTGTGCCGGGTAGCTTGCTCAG |
| | | | TCAGTCAATTGCCAAAGGCTTGACTTATTCAATCGATCA |
| | | | ACATCTGAAACGTACTAACCAAGCGTTAATCGACACGC |
| | | | TCTCGGAACGCGAAGCTCAAATTTTTCAACTGGTGATT |
| | | | GCAGGCAACACCAACAAACAGATGGCTAACGAGCTTTG |
| | | | CGTGGCTATTCGTACCATTGAGGTTCACCGTAGCAAAC |
| | | | TGATGACCAAACTGGGTGTTAACAACCTGGCTGAACTG |
| | | | GTTAAACTGGCGCCGCTGCTGGCACATAAATCCGAATA |
| | | | A |
| LuxR | 18 | AHL-responsive | ATGAAAAACATAAATGCCGACGACACATACAGAATAA |
| | | transcription factor | TTAATAAAATTAAAGCTTGTAGAAGCAATAATGATATT |
| | | | AATCAATGCTTATCTGATATGACTAAAATGGTACATTGT |
| | | | GAATATTATTTACTCGCGATCATTTATCCTCATTCTATG |
| | | | GTTAAATCTGATATTTCAATCCTAGATAATTACCCTAAA |
| | | | AAATGGAGGCAATATTATGATGACGCTAATTTAATAAA |
| | | | ATATGATCCTATAGTAGATTATTCTAACTCCAATCATTC |
| | | | ACCAATTAATTGGAATATATTTGAAAACAATGCTGTAA |
| | | | ATAAAAAATCTCCAAATGTAATTAAAGAAGCGAAAAC |
| | | | ATCAGGTCTTATCACTGGGTTTAGTTTCCCTATTCATAC |
| | | | GGCTAACAATGGCTTCGGAATGCTTAGTTTTGCACATTC |
| | | | AGAAAAAGACAACTATATAGATAGTTTATTTTTACATG |
| | | | CGTGTATGAACATACCATTAATTGTTCCTTCTCTAGTTG |
| | | | ATAATTATCGAAAAATAAATATAGCAAATAATAAATCA |
| | | | AACAACGATTTAACCAAAAGAGAAAAAGAATGTTTAG |
| | | | CGTGGGCATGCGAAGGAAAAAGCTCTTGGGATATTTCA |
| | | | AAAATATTAGGTTGCAGTGAGCGTACTGTCACTTTCCAT |
| | | | TTAACCAATGCGCAAATGAAACTCAATACAACAAACCG |
| | | | CTGCCAAAGTATTTCTAAAGCAATTTTAACAGGAGCAA |
| | | | TTGATTGCCCATACTTTAAAAATTAATAA |
| LuxCDABE | 19 | Photorhabdus | TCAGCAGGACGCACTGACCATTAAAGAGGAGAAAGGT |
| | | luminescens | ACCATGACTAAAAAAATTTCATTCATTATTAACGGCCA |
| | | luciferase operon | GGTTGAAATCTTTCCCGAAAGTGATGATTTAGTGCAAT |
| | | including RBSs | CCATTAATTTTGGTGATAATAGTGTTTACCTGCCAATAT |
| | | | TGAATGACTCTCATGTAAAAAACATTATTGATTGTAAT |
| | | | GGAAATAACGAATTACGGTTGCATAACATTGTCAATTT |
| | | | TCTCTATACGGTAGGGCAAAGATGGAAAAATGAAGAAT |
| | | | ACTCAAGACGCAGGACATACATTCGTGACTTAAAAAAA |
| | | | TATATGGGATATTCAGAAGAAATGGCTAAGCTAGAGGC |
| | | | CAATTGGATATCTATGATTTTATGTTCTAAAGGCGGCCT |
| | | | TTATGATGTTGTAGAAAATGAACTTGGTTCTCGCCATAT |
| | | | CATGGATGAATGGCTACCTCAGGATGAAAGTTATGTTC |
| | | | GGGCTTTTCCGAAAGGTAAATCTGTACATCTGTTGGCA |
| | | | GGTAATGTTCCATTATCTGGGATCATGTCTATATTACGC |
| | | | GCAATTTTAACTAAGAATCAGTGTATTATAAAAACATC |
| | | | GTCAACCGATCCTTTTACCGCTAATGCATTAGCGTTAAG |
| | | | TTTTATTGATGTAGACCCTAATCATCCGATAACGCGCTC |
| | | | TTTATCTGTTATATATTGGCCCCACCAAGGTGATACATC |
| | | | ACTCGCAAAAGAAATTATGCGACATGCGGATGTTATTG |
| | | | TCGCTTGGGGAGGGCCAGATGCGATTAATTGGGCGGTA |
| | | | GAGCATGCGCCATCTTATGCTGATGTGATTAAATTTGGT |
| | | | TCTAAAAAGAGTCTTTGCATTATCGATAATCCTGTTGAT |
| | | | TTGACGTCCGCAGCGACAGGTGCGGCTCATGATGTTTG |
| | | | TTTTTACGATCAGCGAGCTTGTTTTTCTGCCCAAAACAT |
| | | | ATATTACATGGGAAATCATTATGAGGAATTTAAGTTAG |
| | | | CGTTGATAGAAAAACTTAATCTATATGCGCATATATTA |
| | | | CCGAATGCCAAAAAAGATTTTGATGAAAAGGCGGCCTA |
| | | | TTCTTTAGTTCAAAAAGAAAGCTTGTTTGCTGGATTAAA |
| | | | AGTAGAGGTGGATATTCATCAACGTTGGATGATTATTG |
| | | | AGTCAAATGCAGGTGTGGAATTTAATCAACCACTTGGC |
| | | | AGATGTGTGTACCTTCATCACGTCGATAATATTGAGCA |
| | | | AATATTGCCTTATGTTCAAAAAAATAAGACGCAAACCA |
| | | | TATCTATTTTTCCTTGGGAGTCATCATTTAAATATCGAG |
| | | | ATGCGTTAGCATTAAAAGGTGCGGAAAGGATTGTAGAA |
| | | | GCAGGAATGAATAACATATTTCGAGTTGGTGGATCTCA |
| | | | TGACGGAATGCGACCGTTGCAACGATTAGTGACATATA |
| | | | TTTCTCATGAAAGGCCATCTAACTATACGGCTAAGGAT |
| | | | GTTGCGGTTGAAATAGAACAGACTCGATTCCTGGAAGA |
| | | | AGATAAGTTCCTTGTATTTGTCCCATAATAGGTAAAAGT |
| | | | ATGGAAAATGAATCAAAATATAAAACCATCGACCACGT |
| | | | TATTTGTGTTGAAGGAAATAAAAAAATTCATGTTTGGG |
| | | | AAACGCTGCCAGAAGAAAACAGCCCAAAGAGAAAGAA |
| | | | TGCCATTATTATTGCGTCTGGTTTTGCCCGCAGGATGGA |
| | | | TCATTTTGCTGGTCTGGCGGAATATTTATCGCGGAATGG |
| | | | ATTTCATGTGATCCGCTATGATTCGCTTCACCACGTTGG |
| | | | ATTGAGTTCAGGGACAATTGATGAATTTACAATGTCTA |
| | | | TAGGAAAGCAGAGCTTGTTAGCAGTGGTTGATTGGTTA |
| | | | ACTACACGAAAAATAAATAACTTCGGTATGTTGGCTTC |
| | | | AAGCTTATCTGCGCGGATAGCTTATGCAAGCCTATCTG |
| | | | AAATCAATGCTTCGTTTTTAATCACCGCAGTCGGTGTTG |
| | | | TTAACTTAAGATATTCTCTTGAAAGAGCTTTAGGGTTTG |
| | | | ATTATCTCAGTCTACCCATTAATGAATTGCCGGATAATC |
| | | | TAGATTTTGAAGGCCATAAATTGGGTGCTGAAGTCTTT |
| | | | GCGAGAGATTGTCTTGATTTTGGTTGGGAAGATTTAGCT |
| | | | TCTACAATTAATAACATGATGTATCTTGATATACCGTTT |
| | | | ATTGCTTTTACTGCAAATAACGATAATTGGGTCAAGCA |
| | | | AGATGAAGTTATCACATTGTTATCAAATATTCGTAGTA |
| | | | ATCGATGCAAGATATATTCTTTGTTAGGAAGTTCGCATG |
| | | | ACTTGAGTGAAAATTTAGTGGTCCTGCGCAATTTTTATC |
| | | | AATCGGTTACGAAAGCCGCTATCGCGATGGATAATGAT |
| | | | CATCTGGATATTGATGTTGATATTACTGAACCGTCATTT |
| | | | GAACATTTAACTATTGCGACAGTCAATGAACGCCGAAT |
| | | | GAGAATTGAGATTGAAAATCAAGCAATTTCTCTGTCTT |
| | | | AAAATCTATTGAGATATTCTATCACTCAAATAGCAATA |
| | | | TAAGGACTCTCTATGAAATTTGGAAACTTTTTGCTTACA |
| | | | TACCAACCTCCCCAATTTTCTCAAACAGAGGTAATGAA |
| | | | ACGTTTGGTTAAATTAGGTCGCATCTCTGAGGAGTGTG |
| | | | GTTTTGATACCGTATGGTTACTGGAGCATCATTTCACGG |
| | | | AGTTTGGTTTGCTTGGTAACCCTTATGTCGCTGCTGCAT |
| | | | ATTTACTTGGCGCGACTAAAAAATTGAATGTAGGAACT |
| | | | GCCGCTATTGTTCTTCCCACAGCCCATCCAGTACGCCAA |
| | | | CTTGAAGATGTGAATTTATTGGATCAAATGTCAAAAGG |
| | | | ACGATTTCGGTTTGGTATTTGCCGAGGGCTTTACAACAA |
| | | | GGACTTTCGCGTATTCGGCACAGATATGAATAACAGTC |
| | | | GCGCCTTAGCGGAATGCTGGTACGGGCTGATAAAGAAT |
| | | | GGCATGACAGAGGGATATATGGAAGCTGATAATGAAC |
| | | | ATATCAAGTTCCATAAGGTAAAAGTAAACCCCGCGGCG |
| | | | TATAGCAGAGGTGGCGCACCGGTTTATGTGGTGGCTGA |
| | | | ATCAGCTTCGACGACTGAGTGGGCTGCTCAATTTGGCC |
| | | | TACCGATGATATTAAGTTGGATTATAAATACTAACGAA |
| | | | AAGAAAGCACAACTTGAGCTTTATAATGAAGTGGCTCA |
| | | | AGAATATGGGCACGATATTCATAATATCGACCATTGCT |
| | | | TATCATATATAACATCTGTAGATCATGACTCAATTAAA |
| | | | GCGAAAGAGATTTGCCGGAAATTTCTGGGGCATTGGTA |
| | | | TGATTCTTATGTGAATGCTACGACTATTTTTGATGATTC |
| | | | AGACCAAACAAGAGGTTATGATTTCAATAAAGGGCAGT |
| | | | GGCGTGACTTTGTATTAAAAGGACATAAAGATACTAAT |
| | | | CGCCGTATTGATTACAGTTACGAAATCAATCCCGTGGG |
| | | | AACGCCGCAGGAATGTATTGACATAATTCAAAAAGACA |
| | | | TTGATGCTACAGGAATATCAAATATTTGTTGTGGATTTG |
| | | | AAGCTAATGGAACAGTAGACGAAATTATTGCTTCCATG |
| | | | AAGCTCTTCCAGTCTGATGTCATGCCATTTCTTAAAGAA |
| | | | AAACAACGTTCGCTATTATATTAGCTAAGGAGAAAGAA |
| | | | ATGAAATTTGGATTGTTCTTCCTTAACTTCATCAATTCA |
| | | | ACAACTGTTCAAGAACAAAGTATAGTTCGCATGCAGGA |
| | | | AATAACGGAGTATGTTGATAAGTTGAATTTTGAACAGA |
| | | | TTTTAGTGTATGAAAATCATTTTTCAGATAATGGTGTTG |
| | | | TCGGCGCTCCTCTGACTGTTTCTGGTTTTCTGCTCGGTTT |
| | | | AACAGAGAAAATTAAAATTGGTTCATTAAATCACATCA |
| | | | TTACAACTCATCATCCTGTCGCCATAGCGGAGGAAGCT |
| | | | TGCTTATTGGATCAGTTAAGTGAAGGGAGATTTATTTTA |
| | | | GGGTTTAGTGATTGCGAAAAAAAAGATGAAATGCATTT |
| | | | TTTTAATCGCCCGGTTGAATATCAACAGCAACTATTTGA |
| | | | AGAGTGTTATGAAATCATTAACGATGGTTTTAACAACAG |
| | | | GCTATTGTAATCCAGATAACGATTTTTATAGCTTCCCTA |
| | | | AAATATCTGTAAATCCCCATGCTTATACGCCAGGCGGA |
| | | | CCTCGGAAATATGTAACAGCAACCAGTCATCATATTGT |
| | | | TGAGTGGGCGGCCAAAAAAGGTATTCCTCTCATCTTTA |
| | | | AGTGGGATGATTCTAATGATGTTAGATATGAATATGCT |
| | | | GAAAGATATAAAGCCGTTGCGGATAAATATGACGTTGA |
| | | | CCTATCAGAGATAGACCATCAGTTAATGATATTAGTTA |
| | | | ACTATAACGAAGATAGTAATAAAGCTAAACAAGAGAC |
| | | | GCGTGCATTTATTAGTGATTATGTTCTTGAAATGCACCC |
| | | | TAATGAAAATTTCGAAAATAAACTTGAAGAAATAATTG |
| | | | CAGAAAACGCTGTCGGAAATTATACGGAGTGTATAACT |
| | | | GCGGCTAAGTTGGCAATTGAAAAGTGTGGTGCGAAAAG |
| | | | TGTATTGCTGTCCTTTGAACCAATGAATGATTTGATGAG |
| | | | CCAAAAAAATGTAATCAATATTGTTGATGATAATATTA |
| | | | AGAAGTACCACATGGAATATACCTAATAGATTTCGAGT |
| | | | TGCAGCGAGGCGGCAAGTGAACGAATCCCCAGGAGCA |
| | | | TAGATAACTATGTGACTGGGGTGAGTGAAAGCAGCCAA |
| | | | CAAAGCAGCAGCTTGAAAGATGAAGGGTATAAAAGAG |
| | | | TATGACAGCAGTGCTGCCATACTTTCTAATATTATCTTG |
| | | | AGGAGTAAAACAGGTATGACTTCATATGTTGATAAACA |
| | | | AGAAATTACAGCAAGCTCAGAAATTGATGATTTGATTT |
| | | | TTTCGAGCGATCCATTAGTGTGGTCTTACGACGAGCAG |
| | | | GAAAAAATCAGAAAGAAACTTGTGCTTGATGCATTTCG |
| | | | TAATCATTATAAACATTGTCGAGAATATCGTCACTACTG |
| | | | TCAGGCACACAAAGTAGATGACAATATTACGGAAATTG |
| | | | ATGACATACCTGTATTCCCAACATCGGTTTTTAAGTTTA |
| | | | CTCGCTTATTAACTTCTCAGGAAAACGAGATTGAAAGT |
| | | | TGGTTTACCAGTAGCGGCACGAATGGTTTAAAAAGTCA |
| | | | GGTGGCGCGTGACAGATTAAGTATTGAGAGACTCTTAG |
| | | | GCTCTGTGAGTTATGGCATGAAATATGTTGGTAGTTGGT |
| | | | TTGATCATCAAATAGAATTAGTCAATTTGGGACCAGAT |
| | | | AGATTTAATGCTCATAATATTTGGTTTAAATATGTTATG |
| | | | AGTTTGGTGGAATTGTTATATCCTACGACATTTACCGTA |
| | | | ACAGAAGAACGAATAGATTTTGTTAAAACATTGAATAG |
| | | | TCTTGAACGAATAAAAAATCAAGGGAAAGATCTTTGTC |
| | | | TTATTGGTTCGCCATACTTTATTTATTTACTCTGCCATTA |
| | | | TATGAAAGATAAAAAAATCTCATTTTCTGGAGATAAAA |
| | | | GCCTTTATATCATAACCGGAGGCGGCTGGAAAAGTTAC |
| | | | GAAAAAGAATCTCTGAAACGTGATGATTTCAATCATCT |
| | | | TTTATTTGATACTTTCAATCTCAGTGATATTAGTCAGAT |
| | | | CCGAGATATATTTAATCAAGTTGAACTCAACACTTGTTT |
| | | | CTTTGAGGATGAAATGCAGCGTAAACATGTTCCGCCGT |
| | | | GGGTATATGCGCGAGCGCTTGATCCTGAAACGTTGAAA |
| | | | CCTGTACCTGATGGAACGCCGGGGTTGATGAGTTATAT |
| | | | GGATGCGTCAGCAACCAGTTATCCAGCATTTATTGTTAC |
| | | | CGATGATGTCGGGATAATTAGCAGAGAATATGGTAAGT |
| | | | ATCCCGGCGTGCTCGTTGAAATTTTACGTCGCGTCAATA |
| | | | CGAGGACGCAGAAAGGGTGTGCTTTAAGCTTAACCGAA |
| | | | GCGTTTTGATAGTTGA |

TABLE 2
Plasmids
Table S3. Plasmids

| Identifier | Plasmid | Relevant Features | Source |
|---|---|---|---|
| pMM532 | pZA1D-hrtR | HrtR expressed constitutively from promoter ProD with | This |
| | | RBS2, p15a origin, AmpR | work |
| pMM534 | pZE2-PLhrtO- | LuxCDABE expressed constitutively from promoter | This |
| | luxCDABE | PLhrtO, ColE1 origin, KanR | work |
| pMM549 | pZA1D-hrtR-chuA | HrtR expressed consitutively from promoter ProD with | This |
| | | RBS2, ChuA expressed consitiutively from promoter | work |
| | | J23107, p15a origin, AmpR | |
| pMM627 | pZE2-PLhrtO-lux- | Composite plasmid of pMM534 and pMM549, ColE1 | This |
| | hrtR-RBS2-chuA | origin, KanR | work |
| pMM637 | pZE2-PLhrtO-lux- | HrtR RBS variant of plasmid pMM627 (Strength 1783.6 | This |
| | hrtR-RBS1-chuA | AU), ColE1 origin, KanR | work |
| pMM638 | pZE2-PLhrtO-lux- | HrtR RBS variant of plasmid pMM627 (Strength | This |
| | HrtR-RBS4-chuA | 599195.9 AU), ColE1 origin, KanR | work |
| pMM643 | pZE2-PLhrtO-lux- | HrtR RBS variant of plasmid pMM627 (Strength | This |
| | hrtR-RBS3-chuA | 33545.5 AU), ColE1 origin, KanR | work |
| pMM1157 | pZE2-PLhrtO-lux- | ChuA transciptional unit deletion of plasmid pMM643, | This |
| | hrtR-RBS3 | ColE1 origin, KanR | work |
| pMM1161 | pZE1-LuxR-Plux- | AHL-inducible plasmid; LuxR constitutively expressed | This |
| | luxCDABE | from promoter K176009, LuxCDABE under promoter | work |
| | | Plux, ColE1 origin, AmpR | |
| pMM1162 | pZE2-hrtR-RBS3- | LuxCDABE transcriptional unit deletion of plasmid | This |
| | chuA | pMM643, ColE1 origin, KanR | work |
| pMM1489 | pKD236-4b | ThsS constitutively expressed, p15a origin, SpecR | Daeffler |
| | | | K. N., et |
| | | | al., Mol. |
| | | | Syst. Biol., |
| | | | 2017 Apr. 3; |
| | | | 13(4): 923 |
| pMM1532 | pKD237-3a-3-Lux | ThsR constitutively expressed, LuxCDABE under | This |
| | | control of PphsA, ColE1 origin, CamR | work |

Growth and Induction:

For genetic circuit characterization, overnight cultures were diluted 1:100 in fresh LB and incubated with shaking at 37° C. for 2 hours. Cultures were removed from the incubator and 200 μL of culture was transferred to a 96-well plate containing various concentrations of inducer. The plate was returned to a shaking incubator at 37° C. Following 2 hours of incubation, luminescence was read using a BioTek Synergy H1 Hybrid Reader using a is integration time and a sensitivity of 135. Luminescence values, measured in relative luminescence units (RLUs), were normalized by the optical density of the culture measured at 600 nm. For in vitro kinetic studies, subcultured cells were mixed with inducer in a 96-well plate and immediately placed in the plate reader set at 37° C. without shaking. Luminescence and absorbance was read at 5 minute intervals.

A stock solution of hemin (Sigma) was prepared by dissolving hemin powder in 1M NaOH (Sigma) to a concentration of 25 mM, diluting with double distilled water to a final concentration of 500 μM and sterilizing with a 0.2 μm polyethersulfone (PES) filter. Defibrillated horse blood (Hemostat) was used as the source of blood for most experiments. Blood was lysed by first diluting 1:10 in simulated gastric fluid (SGF) (0.2% NaCl, 0.32% pepsin, 84 mM HCl, pH 1.2) before further dilution in culture media. Stock solutions of sodium thiosulfate (Sigma) and 3-O—C₆-HSL (referred to as acyl homoserine lactone (AHL)) (Cayman Chemical) were made in double distilled water.

Indomethacin Mouse Experiments:

All mouse experiments were approved by the Committee on Animal Care at the Massachusetts Institute of Technology. Specific-pathogen free (SPF), male C57BL/6J mice (8-10 weeks of age) were purchased from Jackson Labs and were housed and handled under conventional conditions. Mice were acclimated to the animal facility 1 week prior to the commencement of experiments. Animals were randomly allocated to experimental groups. Researchers were not blinded to group assignments. Prior to indomethacin experiments, a pilot experiment was conducted to determine the transit rate of bacteria through the mouse gastrointestinal tract (FIG. S5). Overnight cultures of E. coli Nissle were centrifuged at 5000×g for 5 minutes and resuspended in an equal volume of 20% sucrose. Animals were inoculated with 200 μL of bacteria culture (approximately 2×10⁸ CPU) by oral gavage. Fecal pellets were collected 2, 4, 6, 8 and 24 hours' post-gavage, weighed, and homogenized in 1 mL of PBS with a 5 mm stainless steel bead using a TissueLyser II (Qiagen) at 25 Hz for 2 minutes. Samples were centrifuged at 500×g for 30 seconds to pellet large fecal debris. Supernatant was serially diluted in sterile PBS and spot plated on MacConkey agar supplemented with kanamycin. Colonies were enumerated following overnight incubation at 37° C. For luminescence assays, luminescence in fecal homogenate was measured in a Biotek Synergy H1 Hybrid Reader with an integration time of 1 second and a sensitivity of 150. Luminescence values were normalized to stool weight normalized CFU values and reported in RUT/ULT.

For indomethacin experiments, animals were inoculated with blood sensor bacteria and fecal pellets were collected 6 hours later for luminescence analysis and CFU enumeration. Indomethacin (Sigma) solution was prepared by dissolving the compound in absolute ethanol to a concentration of 20 mg/mL. Immediately prior to mouse gavage, the indomethacin stock solution was diluted to 1.25 mg/mL in PBS and 0.2 mL of dilute indomethacin solution was administered to each animal (10 mg/kg). Preparation of indomethacin solution using this method was essential to ensure reliable and reproducible induction of gastrointestinal bleeding. The following morning, gastrointestinal bleeding was confirmed by performing a guaiac test (Hemoccult, Beckman Coulter) on fecal pellets from each animal. All mice administered indomethacin were guaiac positive, whereas those administered a PBS control were uniformly guaiac negative. Subsequently, mice were again administered blood sensor bacteria and fecal pellets were collected 6 hours later for luminescence analysis and CFU enumeration.

Preparation of Capsules:

The electronic component in the capsules consisted of four phototransistor detectors (SFH3710, Osram Opto Semiconductors GmbH), a custom bioluminescence detector chip fabricated in a TSMC 65 nm process (Nadeau P., et al., IEEE, 2017 Mar. 6; doi10.1109/ISSCC.2017.7870406), a microcontroller and radio chip (PIC12LF1840T39A, Microchip Technology Inc.), 22 MHz crystal resonator (7M-22.000MEEQ-T, TXC Corporation), 915 MHz chip antenna (0915AT43A0026, Johanson Technology Inc.), two 220 μF ceramic capacitors (CL32A227MQVNNNE, Samsung Electro-Mechanics America, Inc.), and a 5 mAh lithium manganese button-cell battery (MS621FE-FL11E, Seiko Instruments Inc.). The electronics were soldered onto custom four-layer printed circuit boards (Advanced Circuits Inc.) and two screws were epoxied into mounting holes for later attachment of the plastic cell carriers. The assembly was coated with 4-15 μm of Parylene C to act as a moisture barrier (additional methods describing Parylene C deposition described below). A clear rectangular polycarbonate window (500 μm thickness, Rowland Technologies Inc.) was epoxied above the four phototransistor detectors to provide a flat optical interface. The boards were coated with 1-3 mm of epoxy (20845, Devcon) for mechanical stability and then casted into PDMS capsules 13 mm in diameter (Sylgard 184, Dow Corning).

Parylene C Deposition:

Di-chloro-di-p-xylylene (brand name: diX C) dimer was purchased from Daisan Kasei Co. (now a KISCO partner company). Thin film Parylene C coating was preformed using an in-house pyrolysis CVD coating tool. After loading the capsules, 10 grams of dimer was loaded into a thermal evaporation heater and the system was evacuated to 1.3 μbar. The pyrolysis furnace and all other vacuum components were pre-heated prior to deposition. During deposition the dimer was evaporated between 105° C. to 120° C. in order to maintain a constant deposition rate of around 3 Å/s. Upon reaching the desired thickness the deposition chamber was isolated, the system was cooled, the deposition chamber was vented, and the capsules were removed.

Preparation of Cell Carriers:

Cell carriers were machined or injection-molded in ABS plastic (Protolabs Inc.). Semipermeable membranes (0.22 μm pore size, EIMF22205, Millipore Sigma) were affixed to one side of the cell carriers via heat sealing for 35-45 seconds at 230° C. with a stainless steel die. Rubber gaskets for fluidic sealing were die-cut from 380 μm silicone rubber (86435K13, McMaster-Carr) and epoxied to the opposite side of the cell carriers to provide a seal between the carrier and the optical window during experiments.

System Operation, Packet Transmission and Reception: The NPN phototransistor detectors, which may examples of the detectors in FIG. 2A, were operated in a charge-integration mode using each device's intrinsic capacitance as the charge storage mechanism (measured capacitance, Cₒ=8.7 nF). The collector of each detector was connected to the supply rail of the system and the emitters were connected to the system ground through independent low-leakage switches (one per detector) in the custom integrated circuit, which may be an example of the IMP luminescence chip shown in FIG. 2A. At the beginning of a measurement, the emitters were shorted to the system ground via the switches and device capacitances were charged to the system voltage. Then, switches were opened and emitter voltages would start to increase independently in response to the dark currents and photo currents in each detector.

The custom integrated circuit contained a low-power voltage reference (V_{R}=0.625 V) and local oscillator counter (oscillator period, T_{OSC}=5 ms). In each oscillator cycle, the detector voltages for each channel were compared to the reference voltage and, if the reference was exceeded, a count value was saved corresponding to the number of oscillator cycles required the charge the channel. The on-board microprocessor polled the custom circuit once every 8 seconds to determine whether all four channels had exceeded the reference voltage. Once all were exceeded, the microprocessor read the four counter values through a serial peripheral interface and transmitted a short wireless packet at +1.0 dBm with count data using an on-board transmitter, which may be an example of the radio chip in FIG. 2A. The data were received wirelessly by a 900 MHz radio (CC1120 Evaluation Kit, Texas Instruments Inc.) attached to a laptop and processed offline in Matlab (The Mathworks, Inc.).

Photocurrent Estimation with Temperature and Offset Calibration:

The photocurrent detected by the system was estimated using measured quantities and an algorithm for temperature drift and offset calibration, which is described as follows:

Let there be three potentially luminescing sensor channels with counts denoted by Nᵢ: i={1,2,3}. The time required for the photocurrent stimulated by luminescing cells (I_{PH,i}) and the dark background current intrinsic to the photodetectors (I_{D,i}) to charge the channel capacitance (Cₒ) of a channel (i) to the threshold voltage (V_{R}) was quantized using the number of cycles (Nᵢ) counted by the internal oscillator (period, T_{OSC}). The measured cycles were then used to estimate the photocurrent level. The number of cycles required to charge a sensor channel is given by:

$${N}_{i} = ( \frac{{C}_{o} {V}_{R}}{{T}_{OSC}} ) [ \frac{1}{{I}_{D , i} + {I}_{PH , i}} ] .$$

Let there be one reference channel containing no luminescing cells (I_{PH}=0) with a count denoted by Nᵣ. The number of cycles required to charge the reference is given by:

$${N}_{r} = ( \frac{{C}_{o} {V}_{R}}{{T}_{OSC}} ) [ \frac{1}{{I}_{D , r}} ] .$$

The desired photocurrent signal on a channel (I_{PH,i}) is corrupted by the channel's dark current, which has been modelled as:

I_{D,r}=I_{D,OS,i}·f(T),

by separating a temperature-independent, channel-specific dark current offset (I_{D,OS,i}) from a temperature dependent scaling function [f(T)].

To calibrate the temperature and offset, the counts from each sensor channel were first compared to the reference channel by calculating a relative signal Rᵢ:

$${R}_{i} = \frac{1 / {N}_{i} - 1 / {N}_{r}}{1 / {N}_{r}} = ( \frac{{I}_{D , OS , i} \cdot f ( T )}{{I}_{D , OS , r} \cdot f ( T )} - 1 ) + [ \frac{1}{{I}_{D , OS , r} \cdot f ( T )} ] {I}_{PH , i} .$$

In the first term of Rᵢ, the temperature dependence is cancelled, leaving only a dependence on the relative offsets between channels. This term can denoted as R_{i,OS}. Early segments of the count data can be used for each experiment, prior to induction of luminescence from the whole-cell biosensors (I_{PH,i}=0) to estimate R_{i,OS} for each channel. For all experiments, the samples between 0.2 and 0.3 hours (12 to 18 minutes) were used to estimate R_{i,OS}. By substituting the measured offset (R_{i,OS}), as well as the expression for Nᵣ, the final expression for the estimated photocurrent was obtained in terms of known and measured quantities.

$${I}_{PH , i} = ( \frac{{C}_{O} {V}_{R}}{{T}_{S} {N}_{r}} ) [ {R}_{i} - {R}_{i , OS} ] .$$

This calibration procedure was performed using Matlab software (R2017a, The Mathworks, Inc.).

Optical Calibration:

A green LED (λ=525 nm, WP7083ZGD/G, Kingbright) was first calibrated across four decades of input current using an optical power meter located 30 cm away (PM100D and S130C, Thor Labs Inc.). Three capsules were then placed at the same distance as the power meter and measured across the same LED current conditions. The optical power readings were scaled by the ratio of the area of the phototransistor detectors (0.29 mm²) to the area of the S130C sensor (70.9 mm²) in order to estimate the optical power incident on the detectors.

Mobile Phone “App” for Real-Time Reception and Visualization of Results:

A 900 MHz USB dongle (CC1111 USB Evaluation Module Kit, Texas instruments, Inc.) was attached to an Android mobile phone (Galaxy SIII, SCH-I535, Samsung Electronics Co. Ltd.) running a custom application created in Android Studio (Google, Inc.). Temperature and offset calibration was performed on the phone after receiving the first 18 minutes of data to enable offset calibration and the photocurrent estimate was displayed to the user. The raw data was simultaneously uploaded to a cloud service for later analysis.

In Vitro MBED Experiments:

LB culture media supplemented with or without inducer (500 ppm lysed blood (unless otherwise noted), 1.0 mM thiosulfate, or 100 nM AHL) was pre-warmed for at least 2 hours prior to the start of experiments. For blood sensor experiments, overnight cultures were diluted 1:10 in 2×YTPG (20 g tryptone, 5 g NaCl, 10 g yeast extract, 22 mL of 1 M potassium phosphate monobasic, 40 mL of 1 M potassium phosphate dibasic, 0.2% glucose, pH 7.2) and 15 μL of diluted culture was added to wells in the cell carrier (approximately 10⁶ cells per well). Wild-type E. coli Nissle 1917 was added in the reference channel for all experiments. Blood sensor bacteria were added in triplicates to three wells in a single device and values from these three channels were averaged to obtain a single replicate plotted in FIGS. 2C-2E. Technical replicates are depicted in FIGS. 11A-11C. For thiosulfate and AHL experiments, overnight cultures of ThsRS or LuxR containing cells were subcultured for 2 hours in LB prior to addition to cell carriers. Once all four channel were loaded, the cell carrier was fastened to the capsule and fully submerged in pre-warmed media, Cultures were wrapped several times in thick black fabric to block external light, placed in an incubator at 37° C. and data was collected wirelessly for 2 hours. At the end of the experiment, devices were dissembled and cell carriers were discarded. Capsules were sterilized with 70% ethanol and thoroughly washed with distilled water. Capsules were left to air-dry and re-used for future experiments.

Pig Experiments:

All pig experiments were approved by the Committee on Animal Care at the Massachusetts Institute of Technology. Female Yorkshire pigs (50-95 kg) were obtained from Tufts University and housed under conventional conditions. Animals were randomly selected for the experiments. The animals were placed on a clear liquid diet for 24 hours prior to the experiment with the morning feed held on the day of the experiment. At the time of the experiment, the pigs were sedated with Telazol® (tiletamine/zolazepam 5 mg/kg), xylazine (2 mg/kg) and atropine (0.04 mg/kg). An endoscopic overtube (US endoscopy) was placed in the esophagus under endoscopic (Pentax) visual guidance during esophageal intubation. Prior to deposition of devices, 250 mL of neutralization solution (1% sodium bicarbonate and 0.2% glucose) with or without 0.25 mL of pig blood was administered directly to the stomach through the endoscope. Overnight bacterial cultures were diluted 1:10 in 2×YTPG and 15 μL of diluted culture was added to wells in the cell carriers. Devices were assembled and deposited in the pig gastric cavity via endoscopic overtube. Full submersion in gastric fluid was confirmed by endoscopic observation. For 2 hours, data from deposited capsules was acquired via a 900 mHz radio attached to a laptop or the Android cellular phone. Endoscopic videos and radiographs of capsules inside the pig stomach were acquired. Devices were retrieved from the gastric cavity using a hexagonal snare. A total of 6 animals were included in the experiments; 3 were administered neutralization solution containing blood and 3 served as negative controls. Two devices were deposited per pig, such that each group has a sample size of 6.

Data Analysis, Statistics and Computational Methods:

All data were analyzed using GraphPad Prism version 7.03 (Graph Software, San Diego, Calif., USA, graphpad.com). Sequence analysis was performed using Geneious version 9.1.8 (geneious.com) (Kearse M., et al., Bioinformatics, 2013 Jun. 15; 28(12): 1647-49). As noted, error bars represent the SEM of at least three independent experiments carried out on different days. Significance between groups was determined using an unpaired, two-tailed Student's t-test assuming unequal variance. Fold change or signal-to-noise ratio was determined by dividing the normalized luminescence values (RLU/CFU) of samples treated with the maximal inducer concentration with uninduced samples. Response curves were fit to a Hill function: Y=(BₘₐₓXⁿ)/(Kⁿ+Xⁿ)+C, where X is the inducer concentration, Y is the normalized luminescence output, Bₘₐₓ is the maximum luminescence, K is the threshold constant, n is the Hill coefficient and C is the baseline luminescence.

### Example 1: Development of Heme Biosensor

A biosensor was developed for gastrointestinal bleeding as a proof-of-concept MBED for a clinically relevant biomarker. Bleeding in the gastrointestinal tract can be a result of a wide range of causes, including inflammation, cancer, peptic ulcers, non-steroidal anti-inflammatory drug use, portal vein hypertension, among others (Hearnshaw S. A., et al., Gut, 2011 October; 60(10): 1327-35), While cost-effective fecal occult-blood testing exists (Rockey D. C., et al., N. Engl. J. Med., 1998 Jul. 16; 339(3): 153-59), rapid diagnosis of acute bleeding in the upper gastrointestinal tract requires endoscopic observation or aspiration of gastric fluid (Barkun A., et al., Ann. Intern. Med., 2003 Nov. 18; 139(10): 843-57). Importantly, early diagnosis and appropriate treatment of individuals with upper gastrointestinal bleeding has been found to reduce hospital stays and overall medical costs (Lee J. G., et al., Gastrointest. Endosc., 1999 December; 50(6): 755-61). Blood sensing MBEDs could offer an additional means of diagnosing upper gastrointestinal bleeds or monitoring patients at high risk for re-bleeding following endoscopic therapy (Cheng C. L., et al., Dig. Dis. Sci., 2010 September; 5(9): 2577-83) to aid in the triage of individuals who may require further endoscopic or surgical intervention.

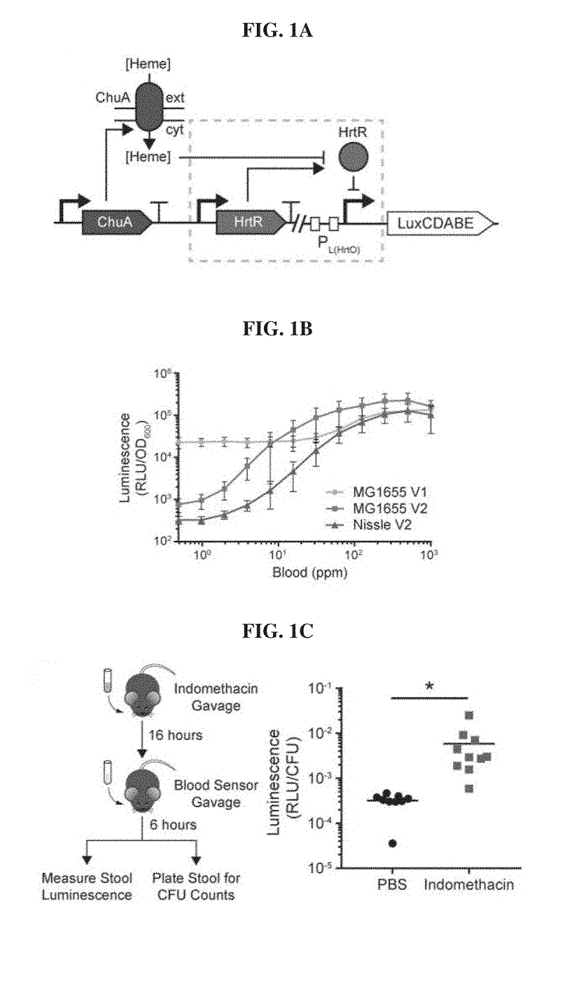

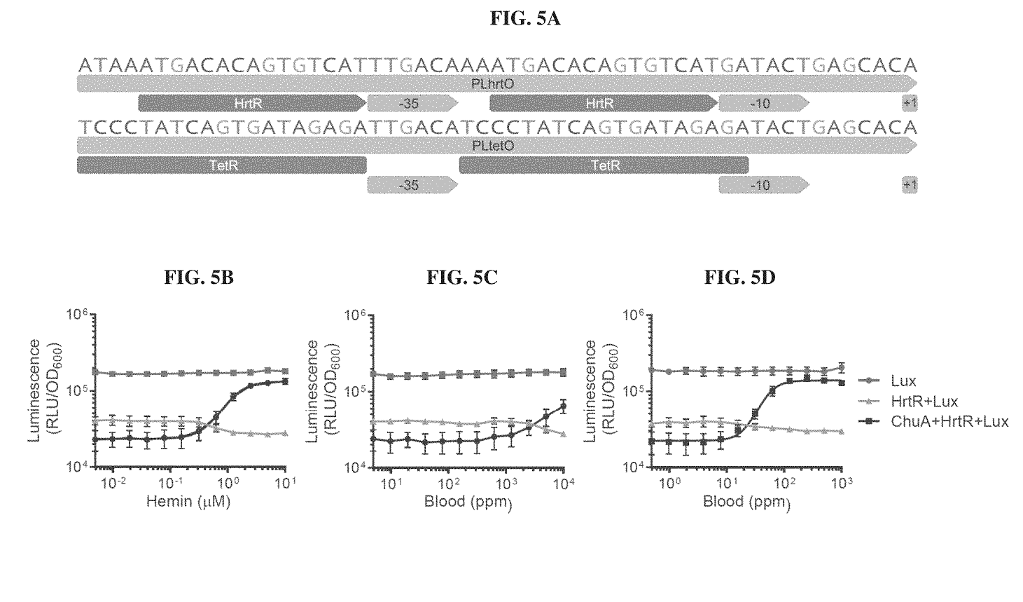

As a bleeding event leads to an accumulation of free heme liberated from lysed red blood cells, the literature was examined for bacterial transcription factors responsive to heme. Lactococcus lactis encodes a heme-regulated TetR-family transcriptional repressor, HrtR, which naturally controls expression of an efflux pump to control intracellular heme-mediated toxicity (Lechardeur D., et al., J. Biol. Chem., 2012 Feb. 10; 287(7): 4752-58). In the absence of heme, HrtR binds to cognate palindromic HrtO operator sequences in the P_{hrtRAB} promoter, repressing promoter activity (FIG. 1A). Conformational changes in HrtR upon heme binding abrogate DNA binding and lead to downstream gene expression (Sawai H., et al., J. Biol. Chem., 2012 Aug. 31; 287(36): 30755-68). To adapt the native P_{hrtAB} promoter to an Escherichia coli chassis, a synthetic promoter was created, P_{L(HrtO)}, based on the strong late promoter of bacteriophage lambda with HrtO operator sequences directly upstream of the −35 and −10 boxes (FIG. 5A). Although photon flux is lower than eukaryotic luciferases, the Phoiorhabdus luminescens luxCDABE luciferase operon was used as the output of P_{L(HrtO)} as it functions at body temperature and encodes all components necessary for intracellular substrate production, thus obviating the need for exogenous substrate (Close D., et al., Sensors, 2012; 12(1): 732-52). Co-transformation of P_{L(HrtO)}-luxCDABE with a constitutively expressing HrtR construct in E. coli MG1655 led to a 4.4-fold reduction in luminescence, indicating HrtR-mediated repression of P_{L(HrtO)} (FIG. 59). However, luminescence levels remained constant irrespective of heme concentration, suggesting that heme could not penetrate the Gram-negative cell envelope. Pathogenic strains of E. coli have evolved heme scavenging systems to acquire scarcely available iron during infection (Torres A. G. and Payne S. M., Mol. Microbiol., 1997 February; 23(4): 825-33). It was hypothesized that introducing the ChuA heme transporter from E. coli O157:H7 into the gene circuit would allow for the transit of extracellular heme into the periplasm, where it could subsequently interact with other cellular components to enter cytoplasm and finally complex with HrtR (FIG. 1A) (Nobles C. L., et al., J. Microbiol. Methods., 2015 November; 118: 7-17). Expression of both HrtR and ChuA yielded a biosensor (MG1655 V1) that responded to increasing heme input with luminescence output with a signal-to-noise ratio (SNR) of 5.9 and a K_{D} of 1 μM heme (FIG. 5B). Luminescence production was also induced by whole horse blood (FIG. 5C) and lysis of red blood cells in simulated gastric fluid greatly improved the sensitivity of detection by liberating heme (K_{D}=115 ppm blood) (FIG. 1B; FIG. 5D).

### Example 2: Optimization of Heme Genetic Circuit

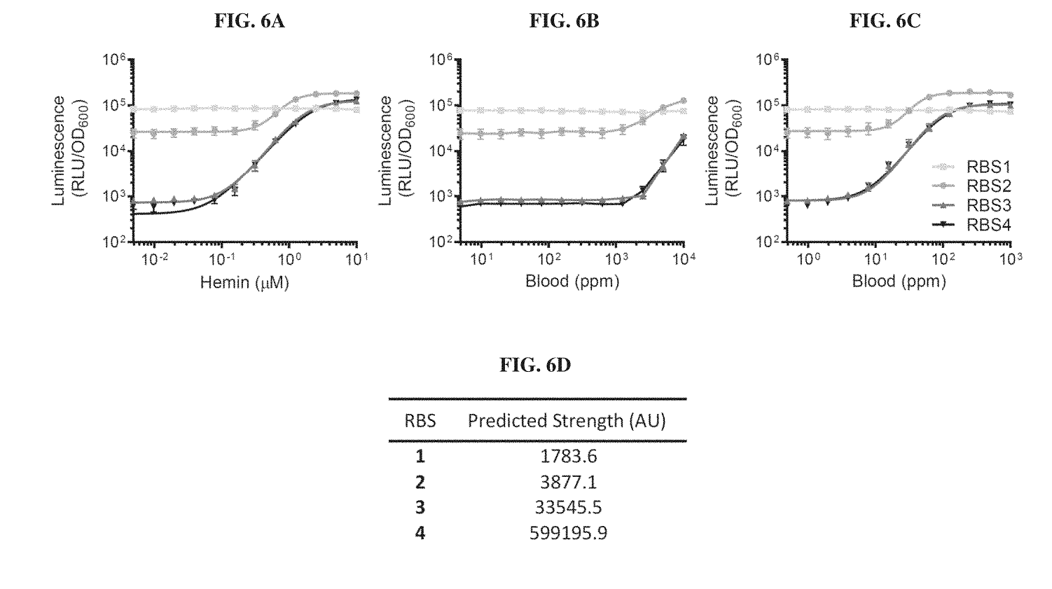

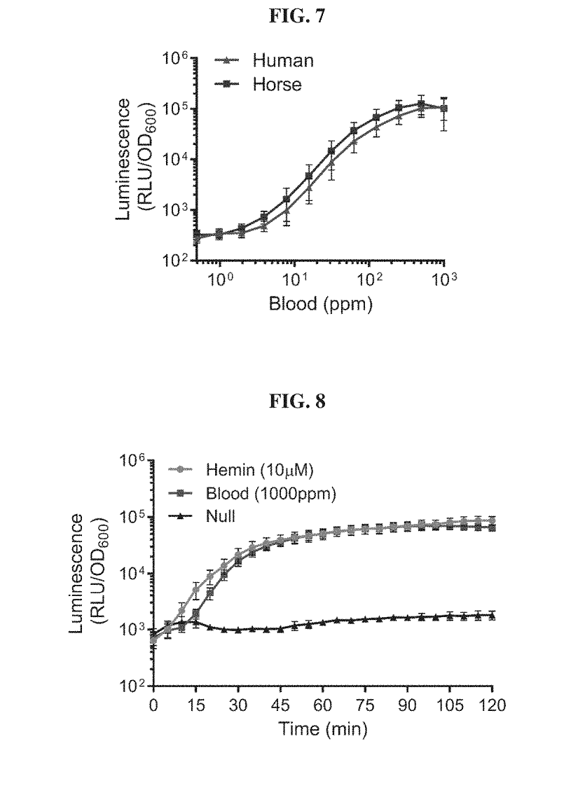

Next, the prototype genetic circuit was iteratively optimized with the goal of improving SNR without compromising maximum luminescence output. Genetic components were combined onto a single high-copy plasmid to minimize plasmid burden as well as the risk of plasmid loss. Increasing the translation initiation strength of HrtR using computationally designed ribosome binding site (RBS) sequences (Salis H. M., et al., Nat. Biotechnol., 2009 October; 27(10): 946-50) decreased baseline luminescence and improved SNR to 132 (MG1655 V2; FIG. 1B; FIGS. 6A-6D). Variations in promoter sequence, number and position of HrtO operator sites in P_{L(HrtO)}, as well as ChuA RBS strength did not lead to appreciable improvements in gene circuit performance. The final gene circuit was transferred to the probiotic E. coli Nissle 1917 strain (Nissle V2) and retained similar performance characteristics compared to the laboratory strain in response to lysed horse blood (SNR=310; K_{D}=95 ppm) (FIG. 19) as well as human blood (FIG. 7). Luminescence was induced rapidly, reaching half-maximal levels within 45 minutes of exposure to heme or lysed blood (FIG. 8).

### Example 3: Demonstration of Optimized Heme Biosensor Functionality

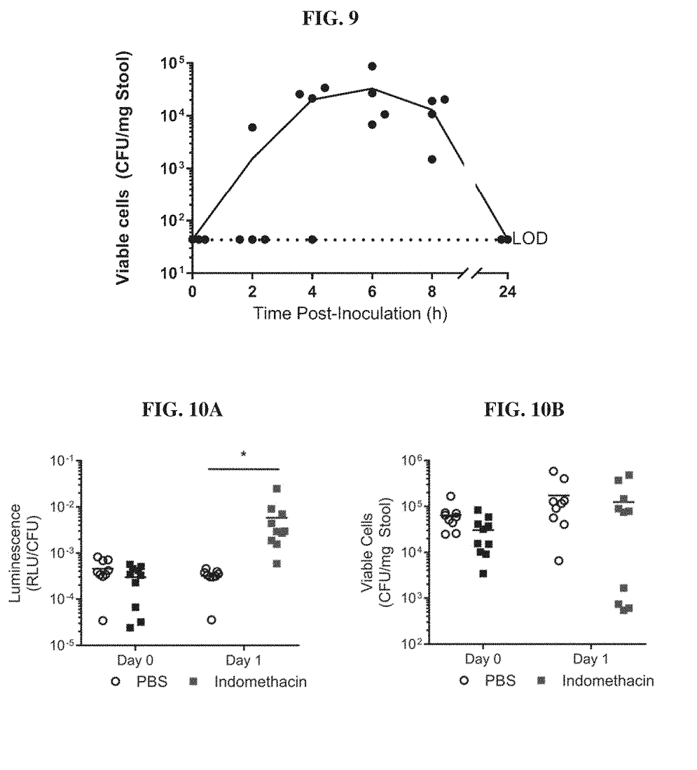

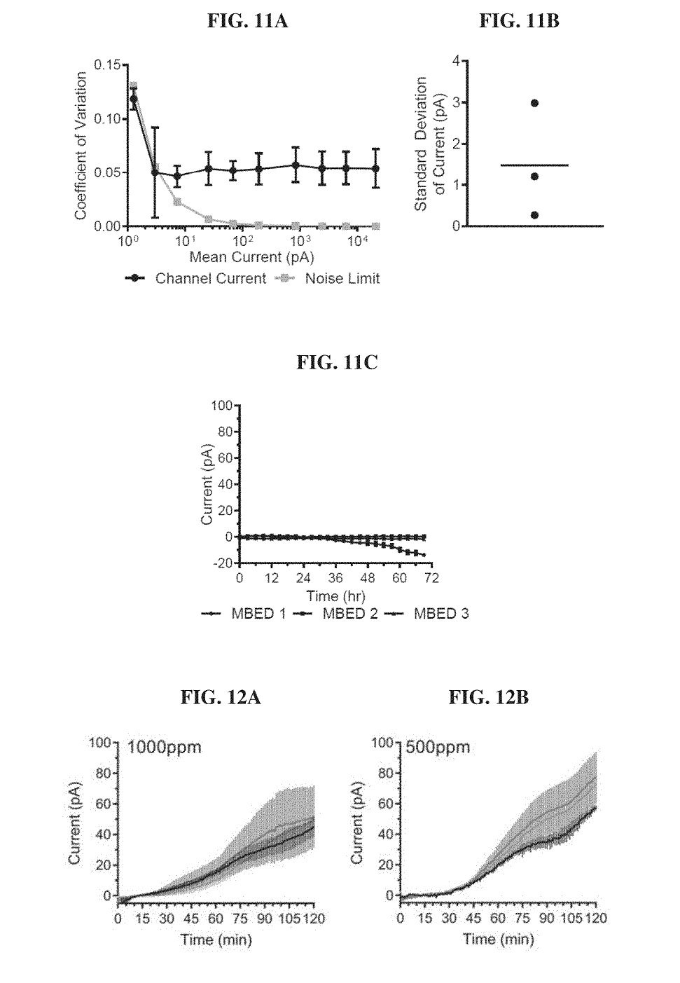

To examine functionality of the bacterial blood sensor in vivo, a murine model of indomethacin-induced gastrointestinal bleeding was employed. Gastroduodenal ulceration is a common adverse effect of non-steroidal anti-inflammatory drug use, as decreased prostaglandin production leads to a thinning of the gastric mucosa and acidification of gastric contents (Lancs A. and Chan F. K. L., Lancet., 2017 Aug. 5; 390(10094): 613-24). Upper gastrointestinal bleeding elicited by oral indomethacin administration could be detected by bacterial blood sensors passing through the gut and measured by observing luminescence activity in fecal pellets (FIG. 1C). Bacterial transit to stool was maximal 6 hours post-inoculation and the blood sensor bacteria could not be recovered from mouse stool 24 hours after administration, suggesting that the engineered strains did not appreciably colonize the murine gut (FIG. 9). At baseline, administration of blood sensor bacteria did not lead to detectable luminescence activity in stool, indicating that the basal heme levels in the murine gut are insufficient to activate the gene circuit (FIGS. 10A-10B). Oral administration of indomethacin generated overt gastrointestinal bleeding overnight as noted by black, tarry stool and positive guaiac tests. Mice subsequently inoculated with blood sensor bacteria demonstrated 18-fold higher luminescence values in fecal pellets as compared to controls (FIG. 1C). Biosensor detection events were fully concordant with guaiac tests and could perfectly discriminate between indomethacin treated and untreated animals. The biosensor can thus effectively detect the presence of gastrointestinal bleeding in vivo.

### Example 4: Integrating Biosensors with Electronic Sensors and Wireless Transmission

Ways of integrating the bacterial biosensor with an electronic sensor and wireless transmission platform were then investigated. Interrogation of cellular bioluminescence is typically performed by power and area-expensive lab equipment that is ill-suited for in situ measurements in the body. Prior demonstrations of custom sensitive bioluminescence detection electronics have required external wiring and have been limited to bench-top assays (Nadeau P., et al., WEE, 2017 Mar. 6; doi10.1109/ISSCC.2017.7870406; Eltoukhy H., et al., IEEE J. Solid-State Circuits, 2006 April; 41(3): 651-61; 36. Singh R. R., et al., IEEE J. Solid-State Circuits, 2012 November; 47(11): 2822-33). For this reason, the first miniaturized, fully-integrated, wireless readout capsule for targeted in vivo sensing of small molecules in the gastrointestinal tract was developed (FIG. 2A). The system encapsulates the previously described nanowatt-level time-based luminometer (Nadeau P., et al., IEEE, 2017 Mar. 6; doi10.1109/ISSCC.2017.7870406), with a microprocessor and wireless transmitter, and provides containment for engineered cells for molecular sensing. The MBED consists of two parts: (1) a molded capsule containing the electronic components, and (2) a plastic carrier for containing cells in one of four cavities. Bioluminescence from activated cells is detected by phototransistors located below each cavity and converted to a digital code using the low-power luminometer chip. In each MBED, one channel acts as a reference to calibrate for background light and temperature-induced dark current variation, while the remaining three are used for independent measurements. Incident photocurrent is supplied to an on-board microcontroller and 900 MHz wireless radio for transmission outside the body. A small button-cell battery (5 mAh) powers the device and the extrapolated MBED power consumption (TABLE 3) suggest a nominal device shelf-life of over 9 months and active operation time of 1.5 months on a full charge. The low power consumption achieved also could allow for battery-free operation in the gastrointestinal tract, using energy harvested from gastric acid (Nadeau P., et al., Nat. Biomed. Eng., 2017; 1: pii: 0022) (33). In addition, two 220 μF ceramic capacitors supplied the instantaneous peak energy required by the radio transmitter. Electronic components were coated in Parylene-C to provide necessary humidity resilience for the sensitive picoampere-level photocurrent measurements. Devices were subsequently encapsulated with a rigid epoxy for mechanical robustness, followed by a molded PMDS capsule for biological compatibility. This multi-layered electronics packaging strategy allows for the creation of a robust cm-scale wireless capsule that, when paired with biosensor cells, can perform continuous, minimally-invasive sensing in vivo.

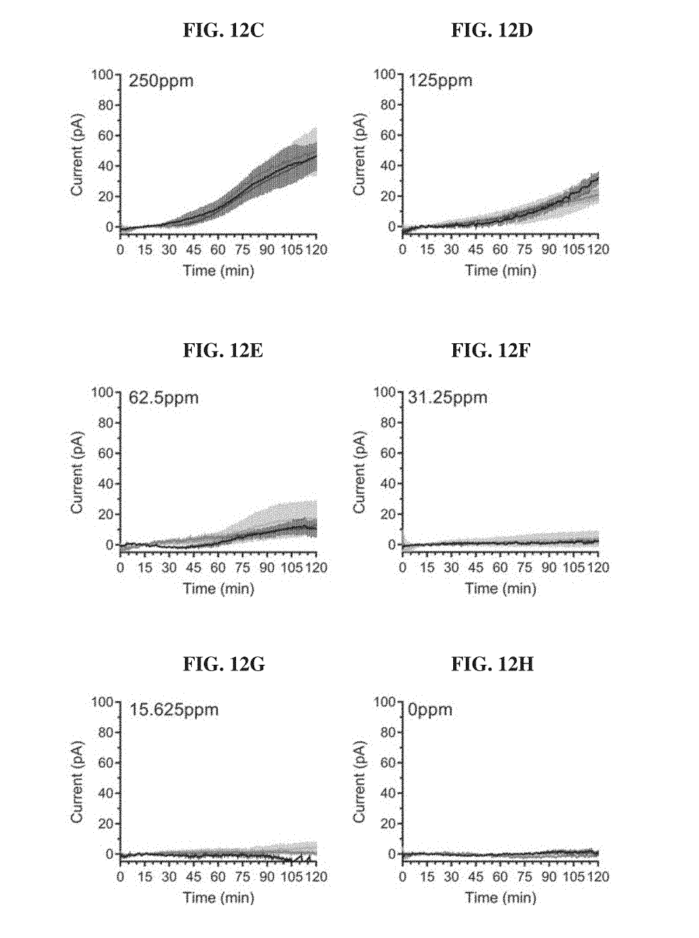

The electronic system is highly sensitive and captured photon flux down to 5×10⁴ photons/s incident on the 0.29 mm² area of the detectors (white-noise limited coefficient of variation 13%ᵣₘₛ, FIG. 2B and FIG. 11A). The mean channel mismatch was less than 6%ᵣₘₛ (FIG. 11A) and mean temperature-induced drift across 5° C. variation was less than 2 pA (FIG. 11B). In addition, MBEDs were stable in simulated gastric fluid for up to 36 h (FIG. 11C), providing sufficient time to perform an ingestible measurement during gastrointestinal transit. To demonstrate integration of the ingestible luminometer capsule and engineered biosensors, the probiotic blood sensor strains were tested in an MBED in vitro. Upon exposure to 500 ppm blood, induced bioluminescence could be observed as soon as 30 minutes (FIG. 2C). This slight delay as compared to plate-reader measurements (FIG. 8) likely owes to diffusion time of heme into the cell cavities. The dose-response curve of blood sensor MBEDs was similar to plate-reader measurements (SNR=76; K_{D}=135 ppm; compare FIG. 2D and FIGS. 12A-12H), with saturation achieved at 250 ppm and significant detection down to 32.5 ppm blood (Student's t-test; p=0.03). Together, MBEDs serve as a flexible platform for sensitive detection of bleeding in fluidic environments.

TABLE 3
Average current consumption of the capsule system. The System Leakage is the static current consumed with all functions of the capsule disabled. The commercial Microcontroller average consumption arises from polling of the luminescence chip every 8 seconds to determine whether a measurement has been completed. The ULP Luminescence Chip consumption results from the continuous operation of the luminescence quantification circuitry. The Wireless consumption results from the transmission of packets. The commercial wireless transmitter dominates the total system consumption (84.4%), whereas the custom illuminometer consumes only a small fraction of the total (<0.2%). Running from the 5 mAh button cell, the system can be expected to last for over 9 months in sleep mode, and for 1.5 months uring continuous active operation.
Current Consumption
System (excluding wireless)

| | |
|---|---|
| System Leakage | 0.30 μA |
| Microcontroller | 0.42 μA |
| ULP Luminescence Chip | 0.01 μA |

Wireless

| | | |
|---|---|---|
| Active Wireless Current | 16.5 | mA |
| Packet Bits | 396 | bits |
| Bit Rate | 50 | kbps |
| Packet Time | 5.92 | ms |
| Sampling Interval | 25 | s |

| | |
|---|---|
| Duty Cycle | 2.4 × 10−4 |

| | |
|---|---|
| Average Wireless Current | 3.96 μA |

| | |
|---|---|
| Total | 4.69 μA |

### Example 5: Demonstration of MBED Adaptability

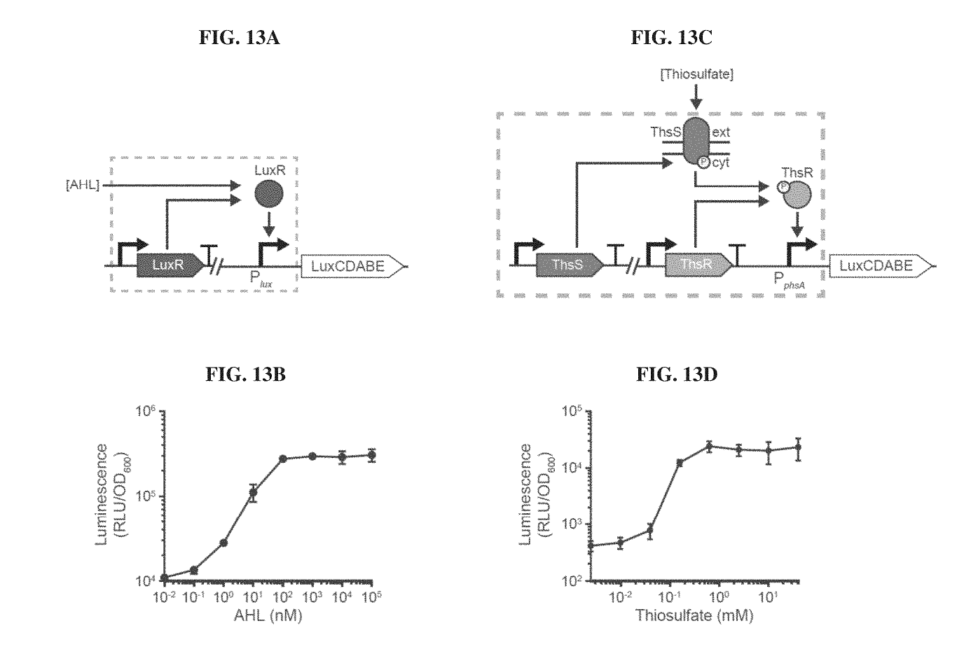

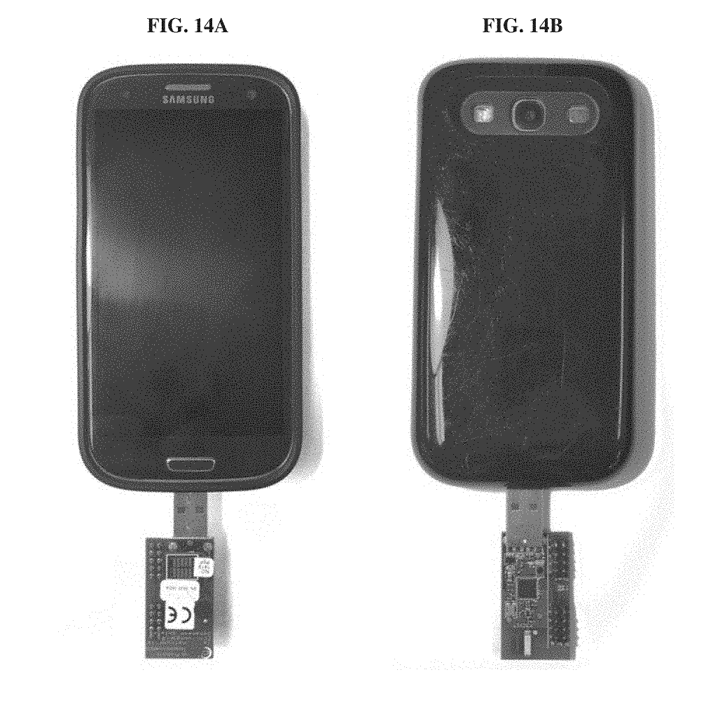

The sensing functionality of MBEDs can be readily adapted to alternative biomarkers. To illustrate this, thiosulfate and acyl-homoserine lactone (AHL) sensors were developed in bacteria to act as bioluminescent reporters. Thiosulfate could serve as a biomarker of gut inflammation as it is elevated in murine models of colitis (Daeffler K. N., et al., Mol. Syst, Biol., 2017 Apr. 3; 13(4): 923). AHLs are molecular signatures of particular bacteria used to coordinate gene expression across populations and their detection in the context of the gut microbiota can indicate the presence of commensal or infectious agents in the gut (Hwang I. Y., et al., Nat. Commun., 2017 Apr. 11; 8: 15028; Schuster M., et al., Annu. Rev. Microbiol., 2013; 67: 43-63; Balagadde F. K., et al., Mol. Syst. Biol., 2008; 4: 187). Thiosulfate- and AHL-inducible genetic circuits were introduced into E. coli Nissle and exposure to increasing concentrations of inducer led to increasing levels of bioluminescence (FIGS. 13A-13D). When integrated with MBEDs, biosensing of different analytes was readily detectable in a fluidic environment (FIG. 2E). As synthetic biologists continue to develop additional biosensors of clinically-relevant gut biomarkers, the breadth of potential analytes of the MBED platform will continue to grow.

### Example 6: Demonstration of MBED Functionality

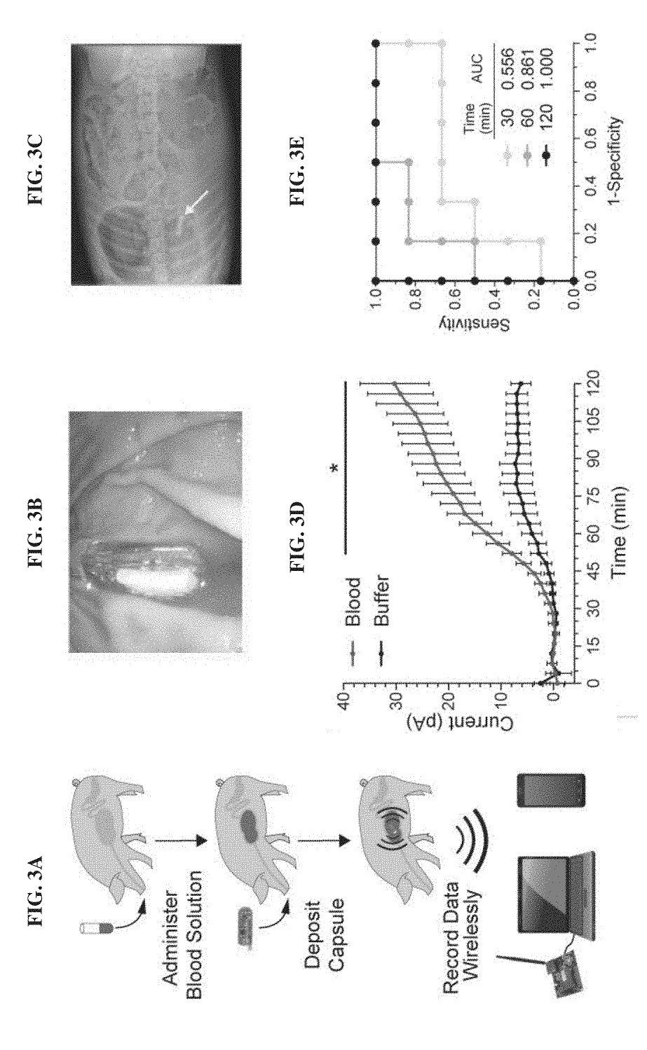

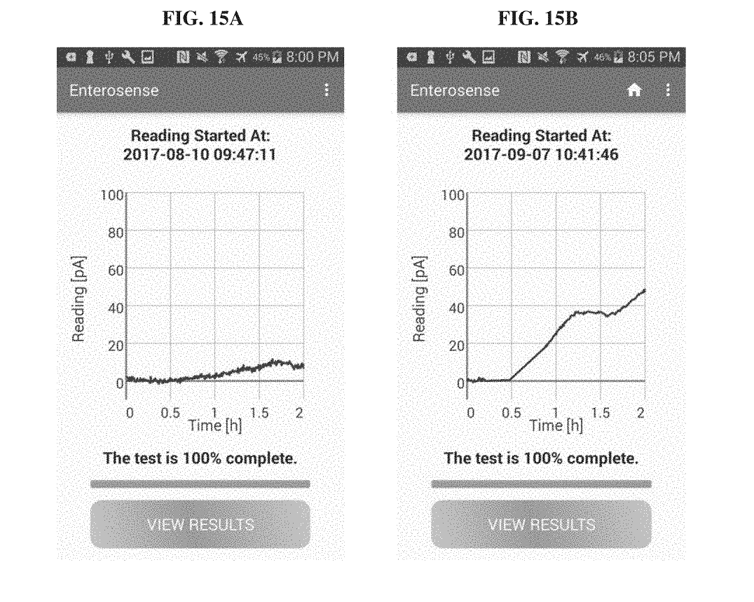

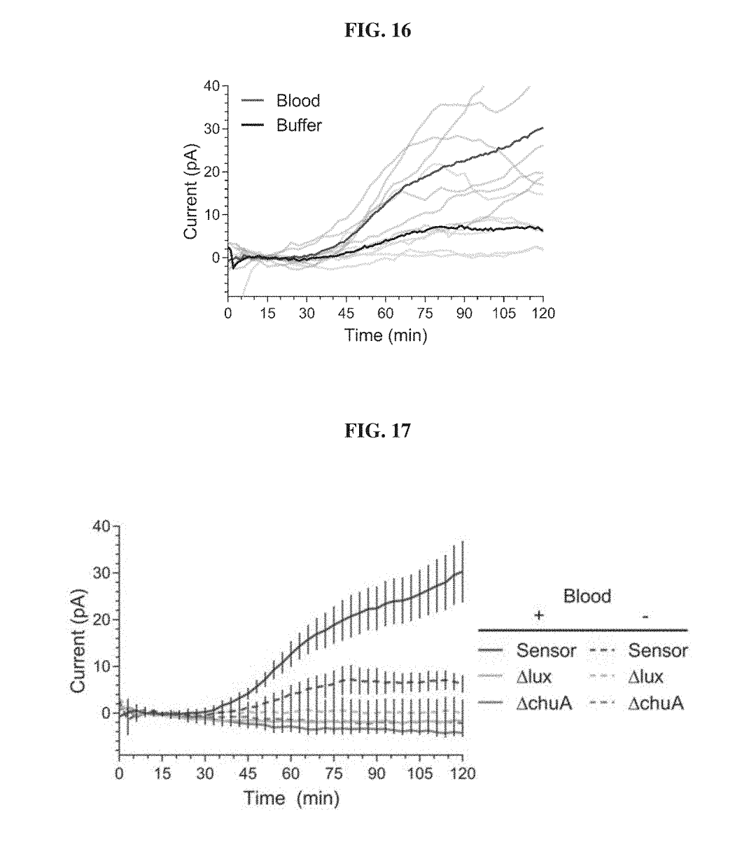

To examine wireless in situ detection of biomolecules with whole-cell biosensors, a blood sensor MBEDs was deployed in a porcine model of gastrointestinal bleeding. Prior to device deposition, pigs were administered a bicarbonate-glucose neutralization solution with or without 0.25 mL of blood (FIG. 3A). The blood sensor MBED was subsequently deposited into the stomach via orogastric tube (FIGS. 3B and 3C). Photocurrent data was wirelessly transmitted from the stomach over the course of 2 hours to a wireless receiver outside of the animal and logged on a laptop computer. In parallel, reception was demonstrated on an Android phone equipped with a 900 MHz wireless receiver dongle and custom application for real-time data processing and visualization (FIG. 14 and FIGS. 15A-15B). The presence of blood in the porcine gastric environment could be observed as early as 52 minutes (Student's t test; p<0.05) and led to a 5-fold increase in photocurrent after 120 minutes as compared to animals given buffer alone (FIG. 3D; FIG. 16). Luminescence production was not detected in biosensors lacking the ChuA heme transporter or the luciferase operon, indicating that observed light production was dependent on a functional genetic circuit activated in the presence of heme (FIG. 17). The receiver operating characteristic of the blood sensing MBED improved over time, with a sensitivity and specificity of 83.3% at 60 minutes and perfect detection at 120 minutes (FIG. 3E). MBEDs can thus detect low-levels of analyte in the gastric environmental with high specificity and sensitivity.

### REFERENCES

1. Balagadde F. K., Song H., Ozaki J., Collins C. H., Barnet M., Arnold F. H., Quake S. R., and You L., A synthetic Escherichia coli predator-prey ecosystem. Mol. Syst. Biol., 2008; 4: 187.
2. Barkun A., Bardou M., and Marshall J. K., Clinical Guidelines Consensus Recommendations for Managing Patients with Nonvariceal Upper Gastrointestinal Bleeding. Ann. Intern. Med., 2003 Nov. 18; 139(10): 843-57.
3. Borkowski O., Ceroni F., Stan G. B., and Ellis T., Overloaded and stressed: whole-cell considerations for bacterial synthetic biology. Curr. Opin. Microbiol., 2016 October; 33: 123-30.
4. Brophy J. A. and Voigt C. A., Principles of genetic circuit design. Nat. Methods., 2014 May; 11(5): 508-20.
5. Cheng C. L., Lin C. H., Kuo C. J., Sung K. F., Lee C. S., Liu N.J., Tang J. H., Cheng H. T., Chu Y. Y., and Tsou Y. K., Predictors of rebleeding and mortality in patients with high-risk bleeding peptic ulcers. Dig. Dis. Sci., 2010 September; 5(9): 2577-83.
6. Chung C. J., Niemela S. L., and Miller R. H., One-step preparation of competent Escherichia coli: transformation and storage of bacterial cells in the same solution. Proc. Natl. Acad. Sci. U.S.A, 1989 April; 86(7): 2172-75.
7. Close D., Xu T., Smartt A., Rogers A., Crossley R., Price S., Ripp S., and Sayler G., The evolution of the bacterial luciferase gene cassette (lux) as a real-time bioreporter. Sensors., 2012; 12(1): 732-52.
8. Courbet A., Endy D., Renard E., Molina F., and Bonnet J., Detection of pathological biomarkers in human clinical samples via amplifying genetic switches and logic gates. Sci. Transl. Med., 2015 May 27; 7(289): 289-83.
9. Daeffler K. N., Galley J. D., Sheth R. U., Ortiz-Velez L. C., Bibb C. O., Shroyer N. F., Britton R. A., and Tabor J. J., Engineering bacterial thiosulfate and tetrathionate sensors for detecting gut inflammation. Mol. Syst. Biol., 2017 Apr. 3; 13(4): 923.
10. Eltoukhy H., Salama K., and El Gamal A., A 0.18-um CMOS Bioluminescence Detection Lab-on-Chip. IEEE J. Solid-State Circuits, 2006 April; 41(3): 651-61.
11. Espah Borujeni A., Channarasappa A. S., and Salis H. M., Translation rate is controlled by coupled trade-offs between site accessibility, selective RNA unfolding and sliding at upstream standby sites. Nucleic Acids Res., 2014 February; 42(4): 2646-59.
12. Gibson D. G., Young L., Chuang R. Y., Venter J. C., Hutchison C. A. 3^{rd}, and Smith H. O., Enzymatic assembly of DNA molecules up to several hundred kilobases. Nat Meth., 2009 May; 6(5): 343-45.
13. Hafezi H., Robertson T. L., Moon G. D., Au-Yeung K. Y., Zdeblick M. J., and Savage G. M., An ingestible sensor for measuring medication adherence. IEEE Trans. Biomed. Eng., 2015 January; 62(1): 99-109.
14. Hearnshaw S. A., Logan Lowe D., Travis S. P., Murphy M. F., and Palmer K. R., Acute upper gastrointestinal bleeding in the UK: patient characteristics, diagnoses and outcomes in the 2007 UK audit. Gut., 2011 October; 60(10): 1327-35.
15. Hwang I. Y., Koh E., Wong A., March J. C., Bentley W. E., Lee Y. S., and Chang M. W., Engineered probiotic Escherichia coli can eliminate and prevent Pseudomonas aeruginosa gut infection in animal models. Nat. Commun., 2017 Apr. 11; 8: 15028.
16. Iddan G., Meron G., Glukhovsky A., and Swain P., Wireless capsule endoscopy. Nature, 2000 May; 405(6785): 417.
17. Kearse M., Moir R., Wilson A., Stones-Havas S., Cheung M., Sturrock S., Buxton S., Cooper A., Markowitz S., Duran C., Thierer T., Ashton B., Meintjes P., and Drummond A., Geneious Basic: an integrated and extendable desktop software platform for the organization and analysis of sequence data. Bioinformatics, 2013 Jun. 15; 28(12): 1647-49.
18. Kotula. J. W., Kerns S. J., Shaket L. A., Siraj L., Collins J. J., Way J. C., and Silver P. A., Programmable bacteria detect and record an environmental signal in the mammalian gut. Proc. Natl. Acad. Sci. U.S.A, 2014 Apr. 1; 111(13): 4838-43.
19. Lanas A. and Chan F. K. L., Peptic ulcer disease. Lancet., 2017 Aug. 5; 390(10094): 613-24.
20. Lechardeur D., Cesselin B., Liebl U., Vos M E., Fernandez A., Brun C., Gruss A., and Gaudu P., Discovery of intracellular heme-binding protein HrtR, which controls heme efflux by the conserved HrtB-HrtA transporter in Lactococcus lactis. J. Biol. Chem., 2012 Feb. 10; 287(7): 4752-58.
21. Lee J. G., Turnipseed S., Romano P. S., Vigil H., Azari R., Melnikoff N., Hsu R., Kirk D., Sokolove P., and Leung J. W., Endoscopy-based triage significantly reduces hospitalization rates and costs of treating upper GI bleeding: a randomized controlled trial. Gastrointest. Enclose., 1999 December; 50(6): 755-61.
22. Lim B., Zimmermann M., Barry N. A., and Goodman A. L., Engineered Regulatory Systems Modulate Gene Expression of Human Commensals in the Gut. Cell., 2017 Apr. 20; 169(3): 547-58.e15.
23. Mimee M., Tucker A. C., Voigt C. A., and Lu T. K., Programming a Human Commensal Bacterium, Bacteroides thetaiotaomicron, to Sense and Respond to Stimuli in the Murine Gut Microbiota. Cell Syst., 2016 Mar. 23; 2(3): 214.
24. Nadeau P., El-Damak D., Glettig D., Kong Y. L., Mo S., Cleveland C., Booth L., Roxhed N., Langer R., Chandrakasan A. P., and Traverso G., Prolonged energy harvesting for ingestible devices. Nat. Biomed. Eng., 2017; 1: pii: 0022.
25. Nadeau P., Mimee M., Carim S., Lu T. K., and Chandrakasan A. P., 21.1 Nanowatt Circuit interface to Whole-Cell Bacterial Sensors. IEEE, 2017 Mar. 6; doi10.1109/ISSCC.2017.7870406.
26. Nobles C. L., Clark J. R., Green S. I., and Maresso A. W., A dual component heme biosensor that integrates heme transport and synthesis in bacteria. J. Microbiol. Methods., 2015 November; 118: 7-17.
27. Norian H., Field Kymissis I., and Shepard K. L., An integrated CMOS quantitative-polymerase-chain-reaction lab-on-chip for point-of-care diagnostics. Lab Chip., 2014 Oct. 21; 14(20): 4076-84.
28. Otis B. and Parviz B., Introducing our smart contact lens project. Google Off. Blog (2014), (available at googleblog.blogspot.com/2014/01/introducing-our-smart-contact-lens.html).
29. Pickard J. M., Maurice C. F., Kinnebrew M. A., Abt M. C., Schenten D., Golovkina T. V., Bogatyrev S. R., Ismagilov R. F., Pamer E. G., Turnbaugh P. J., and Chervonsky A. V., Rapid fucosylation of intestinal epithelium sustains host-commensal symbiosis in sickness. Nature, 2014 Oct. 30; 514(7524): 638-41.
30. Riglar D. T., Giessen T. W., Baym M., Kerns S. J., Niederhuber M. J., Bronson R. T., Kotula J. W., Gerber G. K., Way J. C., and Silver P. A., Engineered bacteria can function in the mammalian gut long-term as live diagnostics of inflammation. Nat. Biotechnol., 2017 July; 35(7): 653-58.
31. Rockey D. C., Koch J., Cello J. P., Sanders L. L., and McQuaid K., Relative frequency of upper gastrointestinal and colonic lesions in patients with positive fecal occult-blood tests. N. Engl. J. Med., 1998 Jul. 16; 339(3): 153-59,
32. Roggo C. and van der Meer J. R., Miniaturized and integrated whole cell living bacterial sensors in field applicable autonomous devices. Curr. Opin. Biotechnol. 2017 June; 45: 24-33.
33. Salis H. M., Mirsky E. A., and Voigt C. A., Automated design of synthetic ribosome binding sites to control protein expression. Nat. Biotechnol., 2009 October; 27(10): 946-50.
34. Sawai H., Yamanaka M., Sugimoto H., Shiro Y., and Aono S., Structural basis for the transcriptional regulation of heme homeostasis in Lactococcus lactis. J. Biol. Chem., 2012 Aug. 31; 287(36): 30755-68.
35. Schuster M., Sexton J. D., Diggle S. P., and Greenberg E. P., Acyl-Homoserine Lactone Quorum Sensing: From Evolution to Application. Annu. Rev. Microbiol., 2013; 67: 43-63.
36. Singh R. R., Leng L., Guenther A., and Genov R., A CMOS-microfluidic chemiluminescence contact imaging microsystem. IEEE J. Solid-State Circuits, 2012 November; 47(11): 2822-33.
37. Slomovic S., Pardee K., and Collins J. J., Synthetic biology devices for in vitro and in vivo diagnostics. Proc. Natl. Acad. Sci. U.S.A, 2015 Nov. 24; 112(47): 14429-35.
38. Torres A. G. and Payne S. M., Haem iron-transport system in enterohaemorrhagic Escherichia coli O157:H7. Mol. Microbiol., 1997 February; 23(4): 825-33.
39. van der Schaar P. J., Dijksman J. F., Broekhuizen-de Gast H., Shimizu J., van Lelyveld N., Lou H., Iordanov V., Wanke C., and Siersema P. D., A novel ingestible electronic drug delivery and monitoring device. Gastrointest. Enclose., 2013 September; 78(3): 520-28.
40. Wang H., Magnetic sensors for diagnostic medicine: CMOS-based magnetic particle detectors for medical diagnosis applications. IEEE Microw. Mag., July 2013; 14(5): 110-30.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases consisting of and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A device comprising an electrical component wherein the electrical component comprises:
at least one detector configured to charge a respective capacitance, wherein each of the at least one detector is configured to detect an output from a biosensor component, optionally wherein at least one detector is a photodetector;
a comparator configured to compare respective voltage signals from each of the at least one detector to a reference voltage, each voltage signal indicating the charge stored by the respective capacitance;
an oscillation counter configured to, when the voltage signal from a first detector of the at least one detector exceeds the reference voltage, store a number of oscillator cycles taken for the first detector to charge the capacitance; and
a transmitter configured to, when the voltage signals from each of the at least one detector exceed the reference voltage, wirelessly transmit the respective stored numbers of oscillator cycles taken for the at least one detector to charge the capacitance.

2. (canceled)

3. The device of claim 1, wherein the device contains a calibration scheme for detecting and removing background light and temperature-induced drift.

4. The device of claim 1, wherein the device is shaped as a capsule or spherocylinder; optionally wherein the capsule or spherocylinder comprises a cross-sectional diameter that is shorter than 10 cm, 9 cm, 8 cm, 7 cm, 6 cm, 5 cm, 4 cm, 3 cm, 2 cm, or 1 cm.

5. (canceled)

6. The device of claim 1, wherein the device can be swallowed by a patient.

7. The device of claim 1, further comprising at least one biosensor component, wherein each of the at least one biosensor component:
is sensitive to the presence of at least one signal analyte; and
communicates the presence of the at least one signal analyte to the electrical component, optionally wherein the communication is proportional to the abundance of the at least one signal analyte; and
optionally wherein each of the at least one biosensor component is separated from the outside environment by a semi-permeable membrane that permits diffusion of the at least one signal analyte.

8. (canceled)

9. The device of claim 7, wherein the semi-permeable membrane is a polyethersulfone membrane filter.

10. The device of claim 7, wherein at least one of the at least one biosensor component is an enzymatic biosensor or a non-enzymatic biosensor; optionally wherein: (i) the non-enzymatic biosensor comprises an antibody, a binding protein, or a nucleic acid and/or (ii) the enzymatic or non-enzymatic biosensor is a cellular biosensor comprising at least one microorganism.

11.-12. (canceled)

13. The device of claim 10, wherein the enzymatic or non-enzymatic biosensor is a cellular biosensor comprises at least one microorganism, wherein the at least one microorganism is present in the device in a dormant state; optionally wherein the at least one microorganism: (i) is combined with additional substances to aid in removing the at least one microorganism from its dormant state, to provide nutrients to the at least one microorganism, and/or to prolong the lifetime of the at least one microorganism; and/or (ii) comprises an engineered genetic circuit.

14.-15. (canceled)

16. The device of claim 13, wherein the output of the engineered genetic circuit is luminescence, fluorescence, ion flow, or turbidity; optionally wherein at least one analyte is selected from the group consisting of a microorganism, a biomolecule, or an inorganic molecule.

17.-18. (canceled)

19. The device of claim 16, wherein at least one signal analyte is a biomolecule selected from the group consisting of heme, thiosulfate, and acyl-homoserine lactone.

20. A method of detecting at least one signal analyte in situ comprising contacting the device of claim 1 with a sample and comparing the output of the device to a control; optionally wherein the sample is selected from the group consisting of soil, water, air, or food.

21. (canceled)

22. A method of monitoring the health of a patient comprising contacting the device of claim 1 with a patient and comparing the output of the device to a control; optionally wherein: (i) the control is established through analysis of a population of healthy patients; (ii) the contacting of the device with the patient occurs by oral administration or deposition of the device in the esophagus, stomach, or intestine; and/or (iii) the contacting of the device with the patient occurs by surgical implantation.

23.-25. (canceled)

26. The method of claim 22, wherein the patient is a human patient; optionally wherein the human patient is predisposed and/or diagnosed to a disease, disorder, morbidity, sickness, or illness.

27.-28. (canceled)

29. A device contained within a capsule or spherocylinder suitable for ingestion comprising an electrical component and at least one biosensor component wherein:
the electrical component comprises wireless low-power electronics powered by (a) a battery, (b) energy harvesting, or (c) wireless power transfer, wherein the low-power electronics comprise at least one detector, optionally wherein at least one detector is a photodetector; and
each biosensor component (a) is separated from the external environment via a semi-permeable membrane, (b) is sensitive to the presence of at least one signal analyte, and (c) communicates the presence of the at least one signal analyte to the electrical component, optionally wherein: (i) the communication is proportional to the abundance of the at least one signal analyte and/or (ii) the semi-permeable membrane is a polyethersulfone membrane filter; and
optionally wherein the capsule or spherocylinder comprises a cross-sectional diameter that is shorter than 10 cm, 9 cm, 8 cm, 7 cm, 6 cm, 5 cm, 4 cm, 3 cm, 2 cm, or 1 cm.

30.-32. (canceled)

33. The device of claim 29, wherein at least one of the at least one biosensor component is an enzymatic biosensor or a non-enzymatic biosensor, optionally wherein: (i) the non-enzymatic biosensor comprises an antibody, a binding protein, or a nucleic acid; and/or (ii) the enzymatic biosensor or non-enzymatic biosensor is a cellular biosensor comprising at least one microorganism.

34.-35. (canceled)

36. The device of claim 33, wherein: (i) at least one microorganism is present in the device in a dormant state; (ii) at least one microorganism is combined with additional substances to aid in removing the at least one microorganism from its dormant state, to provide nutrients to the at least one microorganism, and/or to prolong the lifetime of the at least one microorganism; and/or (iii) at least one microorganism comprises an engineered genetic circuit;
optionally wherein the device further comprises at least one control component comprising a reference microorganism for calibration to remove background light and temperature induced drift.

37.-39. (canceled)

40. The device of claim 36, wherein the output of the engineered genetic circuit is luminescence, fluorescence, ion flow, or turbidity; optionally wherein at least one signal analyte is selected from the group consisting of a microorganism, a biomolecule, or an inorganic molecule.

41.-42. (canceled)

43. The device of claim 42, wherein at least one signal analyte is a biomolecule selected from the group consisting of heme, thiosulfate, and acyl-homoserine lactone.

44. A method of monitoring the health of a patient comprising orally administering the device of claim 29 to a patient and comparing the output of the device to a control; optionally wherein the control is established through analysis of a population of healthy patients.

45. (canceled)

46. The method of claim 44, wherein the patient is a human patient, optionally wherein the human patient is predisposed and/or diagnosed to a disease, disorder, morbidity, sickness, or illness.

47.-48. (canceled)


SEQUENCE LISTING (19 sequences)

SEQ ID NO: 1 - DNA, 15 bp, Artificial Sequence
  HrtR operator sequence
atgacacagtgtcat

SEQ ID NO: 2 - DNA, 55 bp, Artificial Sequence
  Heme-inducible Promoter
ataaatgacacagtgtcatttgacaaaatgacacagtgtcatgatactgagcaca

SEQ ID NO: 3 - DNA, 55 bp, Artificial Sequence
  AHL-inducible promoter
acctgtaggatcgtacaggtttacgcaagaaaatggtttgttatagtcgaataaa

SEQ ID NO: 4 - DNA, 342 bp, Artificial Sequence
  Thiosulfate-inducible promoter
ttcaagcattattatgctgttttttgaagtgaatgtgcggccatctagccgcacattttgcatctaaaacatgcagtcatcagcaaaataataaacttttccccaatatgtggtttaccacaatttacaggaattcactcctgtggtggtgcaaatttgaactgtgaattgcttcacaaacgccgctatcgcaatgtcagtatgtggtttaccacaatatctaatatcactctgctcaataacaatgatgaaaaccttaggaagaagttaattgtgttaaacagttaactaggggctttatctaacgctctcctaaggacaactgtcattgggagatttaac

SEQ ID NO: 5 - DNA, 70 bp, Artificial Sequence
  Constitutive Promoter+RBS for ChuA
tttacggctagctcagccctaggtattatgctagcacatttccaacactaacccaagggagctttaaatc

SEQ ID NO: 6 - DNA, 144 bp, Artificial Sequence
  Consitutive Promoter for HrtR
cacagctaacaccacgtcgtccctatctgctgccctaggtctatgagtggttgctggataactttacgggcatgcataaggctcgtataatatattcagggagaccacaacggtttccctctacaaataattttgtttaacttt

SEQ ID NO: 7 - DNA, 59 bp, Artificial Sequence
  Consitutive Promoter+RBS for LuxR
tttacggctagctcagtcctaggtattatgctagcactagaaaagaggagaaaactaga

SEQ ID NO: 8 - DNA, 68 bp, Artificial Sequence
  Constitutive Promoter+RBS for ThsS
ttgacagctagctcagtcctaggtattgtgctagcctagtatcgatctccataactatcctatagatc

SEQ ID NO: 9 - DNA, 69 bp, Artificial Sequence
  Constitutive Promoter+RBS for ThsR
tttacggctagctcagtcctaggtactatgctagcagaaatataaagaacgatctatttatccgcgtac

SEQ ID NO: 10 - DNA, 35 bp, Artificial Sequence
  HrtR RBS variant
gctataagaaaacaccctttataatctaggttaat

SEQ ID NO: 11 - DNA, 16 bp, Artificial Sequence
  HrtR RBS variant
attaaagaggagaaag

SEQ ID NO: 12 - DNA, 35 bp, Artificial Sequence
  HrtR RBS variant
tatactctaattaatcacataataaggacgaattt

SEQ ID NO: 13 - DNA, 27 bp, Artificial Sequence
  HrtR RBS variant
agccgcaaacatataaggaggaacccc

SEQ ID NO: 14 - DNA, 570 bp, Artificial Sequence
  Heme-responsive transcriptional repressor
atgccaaaatcaacctattttagtctttctgacgaaaaacgaaaacgtgtctatgatgcctgtttactagaatttcaaacgcactctttccatgaagctaaaatcatgcacatcgtaaaagcacttgatatcccaagaggaagtttttatcaatactttgaagatttgaaggattcatactattatatcttgtcacaggaaactgtcgagattcatgatttattttttaatttactaaaagaatatcctctagaagttgctcttaataaatacaagtatcttcttcttgaaaatttagtaaattcgccccaatataatctttataaatatcgatttttagattggacttatgaattagaaagagattggaagcctaaaggcgaggtaactgttcccgctcgtgaacttgataatcctatttcccaagtattaaaatcagtcattcacaatctagtttatcgcatgtttagtgaaaattgggatgaacaaaagtttattgaaacttacgataaagaaatcaaattgctcacagagggcttgcttaattatgttactgaaagcaaaaaatag

SEQ ID NO: 15 - DNA, 1983 bp, Artificial Sequence
  Outer membrane heme transporter
atgtcacgtccgcaatttacctcgttgcgtttgagtttattggccttagctgtttctgccaccttgccaacgtttgcttttgctactgaaaccatgaccgttacggcaacggggaatgcccgtagttccttcgaagcgcctatgatggtcagcgtcatcgacacttccgctcctgaaaatcaaacggctacttcagccaccgatctgctgcgtcatgttcctggaattactctggatggtaccggacgaaccaacggtcaggatgtaaatatgcgtggctatgatcatcgcggcgtgctggttcttgtcgatggtgttcgtcagggaacggataccggacacctgaatggcacttttctcgatccggcgctgatcaagcgtgttgagattgttcgtggaccttcagcattactgtatggcagtggcgcgctgggtggagtgatctcctacgatacggtcgatgcaaaagatttattgcaggaaggacaaagcagtggttttcgtgtctttggtactggcggcacgggggaccatagcctgggattaggcgcgagcgcgtttgggcgaactgaaaatctggatggtattgtggcctggtccagtcgcgatcggggtgatttacgccagagcaatggtgaaaccgcgccgaatgacgagtccattaataacatgctggcgaaagggacctggcaaattgattcagcccagtctctgagcggtttagtgcgttactacaacaacgacgcgcgtgaaccaaaaaatccgcagaccgttggggcttctgaaagcagcaacccgatggttgatcgttcaacaattcaacgcgatgcgcagctttcttataaactcgccccgcagggcaacgactggttaaatgcagatgcaaaaatttattggtcggaagtccgtattaatgcgcaaaacacggggagttccggcgagtatcgtgaacagataacaaaaggagccaggctggagaaccgttccactctctttgccgacagtttcgcttctcacttactgacatatggcggtgagtattatcgtcaggaacaacatccgggcggcgcgacgacgggcttcccgcaagcaaaaatcgattttagctccggctggctacaggatgagatcaccttacgcgatctgccgattaccctgcttggcggaacccgctatgacagttatcgcggtagcagtgacggttacaaagatgttgatgccgacaaatggtcatctcgtgcggggatgactatcaatccgactaactggctgatgttatttggctcatatgcccaggcattccgcgccccgacgatgggcgaaatgtataacgattctaagcacttctcgattggtcgcttctataccaactattgggtgccaaacccgaacttacgtccggaaactaacgaaactcaggagtacggttttgggctgcgttttgatgacctgatgttgtccaatgatgctctggaatttaaagccagctactttgataccaaagcgaaggattacatctccacgaccgtcgatttcgcggcggcgacgactatgtcgtataacgtcccgaacgccaaaatctggggctgggatgtgatgacgaaatataccactgatctgtttagccttgatgtggcctataaccgtacccgcggcaaagacaccgataccggcgaatacatctccagcattaacccggatactgttaccagcactctgaatattccgatcgctcacagtggcttctctgttgggtgggttggtacgtttgccgatcgctcaacacatatcagcagcagttacagcaaacaaccaggctatggcgtgaatgatttctacgtcagttatcaaggacaacaggcgctcaaaggtatgaccactactttggtgttgggtaacgctttcgacaaagagtactggtcgccgcaaggcatcccacaggatggtcgtaacggaaaaattttcgtgagttatcaatggtaa

SEQ ID NO: 16 - DNA, 1785 bp, Artificial Sequence
  Thiosulfate-responsive histidine kinase
atgtcccgcctgctgctgtgtatctgtgttctgctgttctcttctgtggcgtggtctaaaccgcagcagttttatgtgggcgtactggctaactggggtcatcagcaagccgttgaacgttggaccccgatgatggagtatctgaacgaacatgtgccggacgcggaatttcacgtctacccgggcaacttcaaagcactgaacctggcaatggaactgggccagattcagttcattatcactaacccgggccaatatctgtacctgagcaatcagtacccgctgtcttggctggcgaccatgcgttctaagcgtcacgatggtaccacttctgcgatcggttccgccattattgtccgcgcggacagcgactaccgcaccctgtacgacctgaaaggtaaagtggtggctgcgtccgacccgcatgctctgggtggctaccaagcgaccgtcggtctgatgcattccctgggcatggatccggacaccttcttcggtgaaaccaagtttctgggctttccactggatccgctgctgtaccaagttcgtgatggcaacgttgacgcggccattaccccactgtgcactctggaggacatggttgcacgcggcgtactgaaatcttccgattttcgtgtgctgaaccctagccgcccggatggtgtagaatgccagtgctctaccaccctgtacccgaactggtctttcgctgcgactgagtctgtatccaccgaactgtctaaagaaatcacgcaggcactgctggaactgccatccgacagcccggcagctatcaaagcgcaactgaccggctggaccagcccgatctcccaactggcggtaatcaaactgttcaaagagctgcacgtaaaaaccccggactctagccgttgggaagccgttaagaagtggctggaagaaaaccgtcactggggtatcctgtctgttctggtgttcatcattgcaacgctgtatcacctgtggattgaataccgcttccaccaaaaaagctcttctctgatcgaatctgaacgtcagctgaaacagcaagctgttgccctggaacgtctgcaatctgctagcatcgttggtgaaattggtgcgggtctggcccacgagattaatcagccgatcgctgcaattacctcttattctgaaggtggcatcatgcgcctgcaaggtaaagaacaggcggatacggatagctgcatcgaactgctggaaaaaatccacaaacagagcactcgcgcaggcgaagtggtgcaccgcatccgtggtctgctgaaacgtcgtgaagcggtgatggtagatgttaacatcctgaccctggtggaagaatccatcagcctgctgcgtctggagctggcacgtcgcgaaatccagatcaacactcagatcaaaggtgaaccgttcttcattactgccgaccgcgttggcctgctgcaagttctgattaacctgatcaaaaactccctggacgcgatcgctgaatctgataatgcccgttctggtaaaatcaacatcgaactggactttaaagagtaccaggtaaacgtctccatcatcgataacggtccgggcctggcgatggattctgacactctgatggctacgttttacactaccaaaatggatggcctgggcctgggtctggcaatctgccgcgaagttatcagcaaccacgacggccacttcctgctgtccaaccgtgacgacggcgttctgggctgtgtggcaaccctgaatctgaaaaaacgcggttctgaagtgccgatcgaagtctaa

SEQ ID NO: 17 - DNA, 612 bp, Artificial Sequence
  Thiosulfate-responsive response regulator
atgcagcagcaaatcaacggcccggtctacctggtggatgatgatgaagccattatcgactccatcgattttttgatggagggctacggttacaaactgaactcgtttaactgcggcgatcgctttttggcagaagtcgatctcacccaggcaggatgtgtaattctggatgcgcgtatgccaggcttaactggtcctcaggtgcaacagctgctgagcgacgcgaaaagcccgcttgcggtcatcttcctgaccggccatggcgatgttccgatggcggttgatgcgttcaaaaatggcgcgttcgatttctttcaaaaacctgtgccgggtagcttgctcagtcagtcaattgccaaaggcttgacttattcaatcgatcaacatctgaaacgtactaaccaagcgttaatcgacacgctctcggaacgcgaagctcaaatttttcaactggtgattgcaggcaacaccaacaaacagatggctaacgagctttgcgtggctattcgtaccattgaggttcaccgtagcaaactgatgaccaaactgggtgttaacaacctggctgaactggttaaactggcgccgctgctggcacataaatccgaataa

SEQ ID NO: 18 - DNA, 756 bp, Artificial Sequence
  AHL-responsive transcription factor
atgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggaatgcttagttttgcacattcagaaaaagacaactatatagatagtttatttttacatgcgtgtatgaacataccattaattgttccttctctagttgataattatcgaaaaataaatatagcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataa

SEQ ID NO: 19 - DNA, 5838 bp, Artificial Sequence
  Photorhabdus luminescens luciferase operon including RBSs
tcagcaggacgcactgaccattaaagaggagaaaggtaccatgactaaaaaaatttcattcattattaacggccaggttgaaatctttcccgaaagtgatgatttagtgcaatccattaattttggtgataatagtgtttacctgccaatattgaatgactctcatgtaaaaaacattattgattgtaatggaaataacgaattacggttgcataacattgtcaattttctctatacggtagggcaaagatggaaaaatgaagaatactcaagacgcaggacatacattcgtgacttaaaaaaatatatgggatattcagaagaaatggctaagctagaggccaattggatatctatgattttatgttctaaaggcggcctttatgatgttgtagaaaatgaacttggttctcgccatatcatggatgaatggctacctcaggatgaaagttatgttcgggcttttccgaaaggtaaatctgtacatctgttggcaggtaatgttccattatctgggatcatgtctatattacgcgcaattttaactaagaatcagtgtattataaaaacatcgtcaaccgatccttttaccgctaatgcattagcgttaagttttattgatgtagaccctaatcatccgataacgcgctctttatctgttatatattggccccaccaaggtgatacatcactcgcaaaagaaattatgcgacatgcggatgttattgtcgcttggggagggccagatgcgattaattgggcggtagagcatgcgccatcttatgctgatgtgattaaatttggttctaaaaagagtctttgcattatcgataatcctgttgatttgacgtccgcagcgacaggtgcggctcatgatgtttgtttttacgatcagcgagcttgtttttctgcccaaaacatatattacatgggaaatcattatgaggaatttaagttagcgttgatagaaaaacttaatctatatgcgcatatattaccgaatgccaaaaaagattttgatgaaaaggcggcctattctttagttcaaaaagaaagcttgtttgctggattaaaagtagaggtggatattcatcaacgttggatgattattgagtcaaatgcaggtgtggaatttaatcaaccacttggcagatgtgtgtaccttcatcacgtcgataatattgagcaaatattgccttatgttcaaaaaaataagacgcaaaccatatctatttttccttgggagtcatcatttaaatatcgagatgcgttagcattaaaaggtgcggaaaggattgtagaagcaggaatgaataacatatttcgagttggtggatctcatgacggaatgcgaccgttgcaacgattagtgacatatatttctcatgaaaggccatctaactatacggctaaggatgttgcggttgaaatagaacagactcgattcctggaagaagataagttccttgtatttgtcccataataggtaaaagtatggaaaatgaatcaaaatataaaaccatcgaccacgttatttgtgttgaaggaaataaaaaaattcatgtttgggaaacgctgccagaagaaaacagcccaaagagaaagaatgccattattattgcgtctggttttgcccgcaggatggatcattttgctggtctggcggaatatttatcgcggaatggatttcatgtgatccgctatgattcgcttcaccacgttggattgagttcagggacaattgatgaatttacaatgtctataggaaagcagagcttgttagcagtggttgattggttaactacacgaaaaataaataacttcggtatgttggcttcaagcttatctgcgcggatagcttatgcaagcctatctgaaatcaatgcttcgtttttaatcaccgcagtcggtgttgttaacttaagatattctcttgaaagagctttagggtttgattatctcagtctacccattaatgaattgccggataatctagattttgaaggccataaattgggtgctgaagtctttgcgagagattgtcttgattttggttgggaagatttagcttctacaattaataacatgatgtatcttgatataccgtttattgcttttactgcaaataacgataattgggtcaagcaagatgaagttatcacattgttatcaaatattcgtagtaatcgatgcaagatatattctttgttaggaagttcgcatgacttgagtgaaaatttagtggtcctgcgcaatttttatcaatcggttacgaaagccgctatcgcgatggataatgatcatctggatattgatgttgatattactgaaccgtcatttgaacatttaactattgcgacagtcaatgaacgccgaatgagaattgagattgaaaatcaagcaatttctctgtcttaaaatctattgagatattctatcactcaaatagcaatataaggactctctatgaaatttggaaactttttgcttacataccaacctccccaattttctcaaacagaggtaatgaaacgtttggttaaattaggtcgcatctctgaggagtgtggttttgataccgtatggttactggagcatcatttcacggagtttggtttgcttggtaacccttatgtcgctgctgcatatttacttggcgcgactaaaaaattgaatgtaggaactgccgctattgttcttcccacagcccatccagtacgccaacttgaagatgtgaatttattggatcaaatgtcaaaaggacgatttcggtttggtatttgccgagggctttacaacaaggactttcgcgtattcggcacagatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagcttcgacgactgagtgggctgctcaatttggcctaccgatgatattaagttggattataaatactaacgaaaagaaagcacaacttgagctttataatgaagtggctcaagaatatgggcacgatattcataatatcgaccattgcttatcatatataacatctgtagatcatgactcaattaaagcgaaagagatttgccggaaatttctggggcattggtatgattcttatgtgaatgctacgactatttttgatgattcagaccaaacaagaggttatgatttcaataaagggcagtggcgtgactttgtattaaaaggacataaagatactaatcgccgtattgattacagttacgaaatcaatcccgtgggaacgccgcaggaatgtattgacataattcaaaaagacattgatgctacaggaatatcaaatatttgttgtggatttgaagctaatggaacagtagacgaaattattgcttccatgaagctcttccagtctgatgtcatgccatttcttaaagaaaaacaacgttcgctattatattagctaaggagaaagaaatgaaatttggattgttcttccttaacttcatcaattcaacaactgttcaagaacaaagtatagttcgcatgcaggaaataacggagtatgttgataagttgaattttgaacagattttagtgtatgaaaatcatttttcagataatggtgttgtcggcgctcctctgactgtttctggttttctgctcggtttaacagagaaaattaaaattggttcattaaatcacatcattacaactcatcatcctgtcgccatagcggaggaagcttgcttattggatcagttaagtgaagggagatttattttagggtttagtgattgcgaaaaaaaagatgaaatgcatttttttaatcgcccggttgaatatcaacagcaactatttgaagagtgttatgaaatcattaacgatgctttaacaacaggctattgtaatccagataacgatttttatagcttccctaaaatatctgtaaatccccatgcttatacgccaggcggacctcggaaatatgtaacagcaaccagtcatcatattgttgagtgggcggccaaaaaaggtattcctctcatctttaagtgggatgattctaatgatgttagatatgaatatgctgaaagatataaagccgttgcggataaatatgacgttgacctatcagagatagaccatcagttaatgatattagttaactataacgaagatagtaataaagctaaacaagagacgcgtgcatttattagtgattatgttcttgaaatgcaccctaatgaaaatttcgaaaataaacttgaagaaataattgcagaaaacgctgtcggaaattatacggagtgtataactgcggctaagttggcaattgaaaagtgtggtgcgaaaagtgtattgctgtcctttgaaccaatgaatgatttgatgagccaaaaaaatgtaatcaatattgttgatgataatattaagaagtaccacatggaatatacctaatagatttcgagttgcagcgaggcggcaagtgaacgaatccccaggagcatagataactatgtgactggggtgagtgaaagcagccaacaaagcagcagcttgaaagatgaagggtataaaagagtatgacagcagtgctgccatactttctaatattatcttgaggagtaaaacaggtatgacttcatatgttgataaacaagaaattacagcaagctcagaaattgatgatttgattttttcgagcgatccattagtgtggtcttacgacgagcaggaaaaaatcagaaagaaacttgtgcttgatgcatttcgtaatcattataaacattgtcgagaatatcgtcactactgtcaggcacacaaagtagatgacaatattacggaaattgatgacatacctgtattcccaacatcggtttttaagtttactcgcttattaacttctcaggaaaacgagattgaaagttggtttaccagtagcggcacgaatggtttaaaaagtcaggtggcgcgtgacagattaagtattgagagactcttaggctctgtgagttatggcatgaaatatgttggtagttggtttgatcatcaaatagaattagtcaatttgggaccagatagatttaatgctcataatatttggtttaaatatgttatgagtttggtggaattgttatatcctacgacatttaccgtaacagaagaacgaatagattttgttaaaacattgaatagtcttgaacgaataaaaaatcaagggaaagatctttgtcttattggttcgccatactttatttatttactctgccattatatgaaagataaaaaaatctcattttctggagataaaagcctttatatcataaccggaggcggctggaaaagttacgaaaaagaatctctgaaacgtgatgatttcaatcatcttttatttgatactttcaatctcagtgatattagtcagatccgagatatatttaatcaagttgaactcaacacttgtttctttgaggatgaaatgcagcgtaaacatgttccgccgtgggtatatgcgcgagcgcttgatcctgaaacgttgaaacctgtacctgatggaacgccggggttgatgagttatatggatgcgtcagcaaccagttatccagcatttattgttaccgatgatgtcgggataattagcagagaatatggtaagtatcccggcgtgctcgttgaaattttacgtcgcgtcaatacgaggacgcagaaagggtgtgctttaagcttaaccgaagcgtttgatagttga